(19) United States
(12) Patent Application Publication
(10) Pub. No.: US 2010/0104580 A1
(43) Pub. Date: Apr. 29, 2010

(54) Altered Immunogenic Landscape in HIV-1 Envelope Proteins

(75) Inventor: Irwin M. Chaiken, Gladwyne, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Philadelphia Health & Education Corporation D/B/A, Philadelphia, PA (US)

(21) Appl. No.: 12/553,672

(22) Filed: Sep. 3, 2009

Related U.S. Application Data
(60) Provisional application No. 61/093,957, filed on Sep. 3, 2008.

(51) Int. Cl. A61K 39/42; A61K 39/00; A61K 38/02; A61K 38/16; C07K 2/00; A61P 31/18

(52) U.S. Cl. 424/160.1; 424/184.1; 514/2; 514/12; 530/300

(57) ABSTRACT

The present invention provides compositions and methods useful in the prevention and treatment of HIV-1 infection in a host subject. High affinity binding of an allosteric dual antagonist to HIV-1 gp120 induces a conformational change in the gp 120 protein that traps the gp 120 protein in a three-dimensional structure that suppresses its function and exposes novel antigenic epitopes to host immune surveillance.

### STATEMENT REGARDING FEDERALLY SPONSORED RESEARCH OR DEVELOPMENT

This invention was made with Government support under grant No. 5 P01 GM 56550-12, R21 AI 071965-01, and U01 AI 067854-02 awarded by the National Institutes of Health. The U.S. Government therefore has certain rights in this invention.

### BACKGROUND OF THE INVENTION

Human immunodeficiency virus (HIV) is a member of the genus Lentivirus, part of the family of Retroviridae. Two strains, HIV-1 and HIV-2, infect humans, though HIV-1 is more virulent, more readily transmissable, and the cause of the majority of HIV infections globally. Both, however, can lead to acquired immunodeficiency syndrome (AIDS), a condition in humans in which the immune system collapses, leading to life-threatening opportunistic infections and malignacies. HIV infection is not curable, and to date, there is no vaccine available.

HIV infection in humans is now pandemic. As of January 2006, the Joint United Nations Programme on HIV/AIDS (UNAIDS) and the World Health Organization (WHO) estimate that AIDS has killed more than 25 million people since it was first recognized on Dec. 1, 1981, making it one of the most destructive pandemics in recorded history.

HIV-1 is composed of two copies of positive single-stranded RNA that codes for the virus's nine genes enclosed by a conical capsid composed of 2,000 copies of the viral protein p24. The single-stranded RNA is tightly bound to nucleocapsid proteins, p7 and enzymes needed for the development of the virion such as reverse transcriptase, proteases, ribonuclease and integrase. A matrix composed of the viral protein p17 surrounds the capsid ensuring the integrity of the virion particle. This is, in turn, surrounded by the viral envelope which is composed of two layers of phospholipids derived from the membrane of a human cell when a newly formed virus particle buds from the cell. Embedded in the viral envelope are proteins from the host cell and about 70 copies of a complex HIV protein that protrudes through the surface of the virus particle known as Env. The envelope glycoprotein of HIV-1 is a trimer that consists of three gp120 exterior envelope glycoproteins and gp41 transmembrane glycoproteins. This glycoprotein complex enables the virus to attach to and fuse with host target cells to initiate the infectious cycle.

Of the nine genes that are encoded within the RNA genome, three of these genes, gag, pol, and env, contain information needed to make the structural proteins for new virus particles. For example, env codes for a protein called gp160 that is broken down by a viral enzyme to form gp120 and gp41 (Chan et al., 1997, Cell 89: 263-73; Wyatt et al., 1998, Science 280: 1884-8; Tan et al., 1997, Proc Natl Acad Sci U S A 94: 12303-8). The six remaining genes, tat, rev, nef, vif, vpr, and vpu (or vpx in the case of HIV-2), are regulatory genes for proteins that control the ability of HIV to infect cells, replicate, or cause disease. The protein encoded by nef appears necessary for the virus to replicate efficiently, and the vpu-encoded protein influences the release of new virus particles from infected cells. The ends of each strand of HIV RNA contain an RNA sequence called the long terminal repeat (LTR). Regions in the LTR act as switches to control production of new viruses and can be triggered by proteins from either HIV or the host cell.

The primary targets for HIV-1 infection in vivo are CD4⁺ T cells and cells of the monocyte/macrophage lineage (Klatzmann et al., 1984, Nature 312:767-8; Dalgleish et al., 1984, Nature 312:763-7). Viral infection is initiated by the binding of gp120 of HIV-1 to the CD4 antigen on the host T cell surface. The binding of gp120 to CD4 promotes a conformational change in gp120 that increases its affinity for a second host-cell receptor, one of the chemokine receptors, either CCR5 or CXCR4 (Wu et al, 1996, Nature 384: 179-83; Dragic et al., 1996, Nature 381: 667-73). The interaction of gp120 with its receptors is believed to promote further conformational rearrangements in the HIV-1 envelope that drive fusion of the viral and host-cell membranes. Blockage of the interactions between gp120 and cell-surface receptors is an attractive target for the prevention of HIV-1 infection through the inhibition of membrane fusion and viral entry. However, gp120 interacting domains of HIV-1 are able to escape immune surveillance by several mechanisms.

Recent investigations using both in vitro and in vivo assays have demonstrated the potential topical microbicide activity of cyanovirin-N (CV-N), an 11 kD protein originally isolated from the cyanobacteria Nostoc ellipsosporum (Boyd et al., 1997, Antimicro Agents Chemother. 41:1521-1530). CV-N inactivates a broad range of M-tropic and T-tropic strains of HIV-1, SIV, FIV and prevents cell-to-cell transmission of infection (Boyd et al., 1997, Antimicro Agents Chemother. 41:1521-1530). CV-N binds specifically to the highly glycosylated viral envelope protein gp120 and to the functionally analogous SIV proteins sgp130 and sgp140. The epitopes on gp120 responsible for CV-N binding appear to be predominantly high-mannose glycosylation sites of the envelope. Recombinant CV-N blocks HIV-1 BaL infection of human ectocervical explants without cytotoxic effects (Tsai et al., 2004, AIDS Res Hum Retroviruses 20:11-18). Gel formulations of CVN applied rectally to male macaques protected against challenge by the SIV/HIV-1 virus SHIV89.6P (Tsai et al, 2003, AIDS Res Hum Retroviruses 19:535-541). In vivo efficacy has also be shown in a vaginal challenge model with female macaques (Tsai et al., 2004, AIDS Res Hum Retroviruses 20:11-18). CV-N showed no clinically adverse effects in these in vivo assays. However, the production costs and consequent cost per dose are limitations of the usage of CV-N alone as a therapeutic.

A screen of a random peptide phage-display library identified several peptides that bind to HIV-1 envelope glycoprotein gp120 (Ferrer et al., (1999, Virol. 73:5795-5802). One 12-mer, named 12p1, was found to inhibit the interaction between gp120 and four-domain soluble CD4 (4dCD4) and between gp120 and 17b, an HIV neutralizing monoclonal antibody. Recently, a derivative of 12p1 obtained using a stable and chemically accessible azidoproline residue as a basis for side-chain bioconjugation reactions through click chemistry has been reported (U.S. Pat. Publication No. 20060135746; Gopi et al., 2006, Chem Med Chem 1:54-57). Recent peptide derivatives of 12p1 have a greater binding affinity for gp 120, compared to 12p1, and also inhibit strongly the interaction between gp120 and both CD4 and 17b.

Acquired immunodeficiency syndrome (AIDS), the global epidemic caused by HIV-1, has created an urgent need for new therapeutic agents and vaccines useful in the treatment and prevention of HIV infection. The present invention fills this need.

### SUMMARY OF THE INVENTION

In one embodiment, the invention comprises method of obtaining a substantially pure population of neutralizing antibodies that prevent, inhibit, or block HIV-1 gp120 from binding to a CD4 T cell receptor, the method comprising the steps of administering to a subject an effective amount of a composition comprising an allosteric dual antagonist in combination with HIV-1 gp120, where when the composition is administered to the subject, the subject produces neutralizing antibodies that bind to HIV-1 gp120; isolating from the serum of the subject a substantially pure population of neutralizing antibodies. In one aspect, the allosteric dual antagonist is selected from the group consisting of a peptide triazole conjugate, HNG-156, a fragment of HNG-156, UM24, a recombinant L5 cyanovirin-N-12p1 chimera, a small molecule, a peptide, or any combination thereof. In another aspect, the subject is a mammal. In another aspect, the mammal is a human.

In another embodiment, the invention comprises a method of exposing a novel antigenic epitope present on the HIV-1 gp120 to immune surveillance in a subject, the method comprising administering to a subject a pharmaceutical composition comprising an immunogen, wherein the immunogen comprises an allosteric dual antagonist, wherein when the allosteric dual antagonist contacts HIV-1 gp120, the allosteric dual antagonist induces a conformational change in the HIV-1 gp120, thereby exposing a novel antigenic epitope present on HIV-1 gp120 to immune surveillance. In one aspect, the allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof. In another aspect, the subject is a mammal. In still another aspect, the mammal is a human.

Yet another embodiment of the invention comprises a method of preventing HIV-1 infection in a subject, the method comprising administering to a

Another embodiment of the invention comprises a method of preventing HIV-1 infection in a subject, the method comprising administering to the subject a therapeutically effective amount of a pharmaceutical composition comprising an immunogen to a subject exposed to HIV-1 or at risk of being exposed to HIV-1, wherein the immunogen comprises an allosteric dual antagonist. In one aspect the allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof. In another aspect, the allosteric dual antagonist is administered in combination with exogenous HIV-1 gp120. In another aspect the allosteric dual antagonist is administered without exogenous HIV-1 gp120. In still another aspect, the subject is a mammal. In another aspect, the mammal is a human.

Still another embodiment of the invention comprises a method of treating HIV-1 infection in a subject, said method comprising administering to said subject a therapeutically effective amount of a pharmaceutical composition to a subject infected with HIV-1, wherein said composition comprises an allosteric dual antagonist and HIV-1 gp120. In one aspect the allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof. In another aspect, the allosteric dual antagonist is administered in combination with exogenous HIV-1 gp120. In another aspect, the allosteric dual antagonist is administered without exogenous HIV-1 gp120. In another aspect, the subject is a mammal. In still another aspect, the mammal is a human.

In another embodiment, the invention comprises a composition for inhibiting binding of HIV-1 gp120 to a host CD4 receptor, the composition comprising an allosteric dual antagonist covalently linked to gp120. In one aspect, the allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof. In another aspect, the host is a mammal. In another aspect, the mammal is a human. subject a therapeutically effective amount of a pharmaceutical composition comprising an immunogen to a subject exposed to HIV-1 or at risk of being exposed to HIV-1, wherein the immunogen comprises an allosteric dual antagonist. In one aspect, the allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof. In another aspect, the subject is a mammal. In still another aspect, the mammal is a human.

Another embodiment of the invention comprises a method of treating HIV-1 infection in a subject, the method comprising administering to a subject a therapeutically effective amount of a pharmaceutical composition to a subject infected with HIV-1, wherein the compositioni comprises an allosteric dual antagonist and HIV-1 gp120. In one aspect, the allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof. In another aspect, the subject is a mammal. In still another aspect, the mammal is a human.

Yet another embodiment, of the invention comprises a method of treating HIV-1 infection of a subject, the method comprising administering to a subject a therapeutically effective amount of a neutralizing antibody to a subject, wherein the neutralizing antibody neutralizes the interaction of HIV-1 gp120 and CD4 receptors. In one aspect the subject is a mammal. In another aspect, the mammal is a human.

Another embodiment of the invention comprises a method of neutralizing the interaction of HIV-1 gp120 and a CD4 T cell receptor in a subject infected with HIV-1, the method comprising administering to a subject a therapeutically effective amount of a pharmaceutical composition to a subject infected with HIV-1, wherein the composition comprises an allokeric dual antagonist and HIV-1 gp120. In one aspect the allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof. In another aspect, the subject is a mammal. In still another aspect, the mammal is a human.

Yet another embodiment of the invention comprises a method of neutralizing the interaction of HIV-1 gp120 and a CD4 T cell receptor in a subject infected with HIV-1, the method comprising administering to a subject a therapeutically effective amount of a neutralizing antibody to the subject, where the neutralizing antibody neutralizes the interaction of HIV-1 gp120 and the CD4 T-cell receptors. In one aspect, the subject is a mammal. In another aspect, the mammal is a human.

Still another embodiment of the invention comprises a pharmaceutical composition comprising an immunogen, wherein the immunogen comprises an allosteric dual antagonist and an HIV-1 gp120. In one aspect the allosteric dual antagonist selected from the group consisting of a peptide triazole conjugate, HNG-156, a fragment of HNG-156, UM24, a recombinant L5 cyanovirin-N-1p12 chimera, a small molecule, a peptide, or any combination thereof.

Yet another embodiment of the invention comprises a pharmaceutical composition comprising a neutralizing antibody, wherein the neutralizing antibody is made in response to an immunogen of the present invention, where the immunogen comprises an allosteric dual antagonist, wherein the neutralizing antibody neutralizes the interaction of HIV-1 gp120 and a CD4 T cell receptor.

Another embodiment of the invention comprises a method of exposing a novel antigenic epitope present on the HIV-1 gp120 to immune surveillance in a subject, the method comprising administering to the subject a pharmaceutical composition comprising an allosteric dual antagonist, wher when the allosteric dual antagonist contacts the HIV-1 gp120, the allosteric dual antagonist induces a conformational change in the HIV-1 gp120, thereby exposing a novel antigenic epitope present on the HIV-1 gp120 to immune surveillance. In one aspect, the allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof. In another aspect, the allosteric dual antagonist is administered in combination with exogenous HIV-1 gp120. In still another aspect, the allosteric dual antagonist is administered without exogenous HIV-1 gp120. In yet another aspect, the subject is a mammal. In still another aspect, the mammal is a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A through FIG. 1C, is a series of graphs depicting the characterization of UM24 in comparison with HNG-156. FIG. 1A depicts percent antiviral inhibition by HNG-156 (squares) and UM24 (circles) as a function of concentration. FIG. 1B is a graph depicting the per cent (%) CD4 binding in the presence of HNG-156 or UM24 as a function of concentration of the peptides. FIG. 1C is a graph depicting the percent (%) 17b binding in the presence of HNG156 or UM24.

FIG. 2, comprising FIG. 2A and FIG. 2B, is a series of graphs depicting the effect of UM24 and HNG-156 on conformation sensitive and insensitive antibody binding to gp120. FIG. 2A is a bar graph depicting ELISA and SPR competition between UM24/HNG-156 and conformation-sensitive ligands. FIG. 2B is a bar graph depicting binding of linear epitope antibodies to gp120 in the presence of 1 mM (11/4C), 10 uM (38.1a, 11/65a, ID6, F425 B4a1) or 100 uM (F425 b4e8, CRA3, 11/68b, 2D7 and CRA4) UM24.

FIG. 3 comprises a graph depicting the titer of ELISA detected anti-YU2gp120 antibodies in the sera of immunized guinea pigs. HIV-1_{YU2}gp120 was immobilized on ELISA plates, serum dilutions applied, and antibodies detected with anti-guinea pig IgG labeled with Horseradish peroxidase (HRP).

FIG. 4 comprises a graph depicting the inhibition of CD4 binding to HIV-1_{YU2}gp120 by bleed 6 sera. The ability of sera to inhibit the gp120-CD4 interaction was probed by sandwich ELISA. CD4 binding was detected with a biotinylated antibody. The data represent three independent trials with error bars indicating standard deviation.

FIG. 5 comprises a graph depicting ELISA testing the inhibition of 17b binding to YU2gp120 by serum of immunized guinea pigs. YU2gp120 was immobilized and incubated with a mixture of 17b-biotin with serum dilutions. Bound 17b-biotin was detected with streptavidin-HRP.

FIG. 6, comprises FIG. 6A through FIG. 6C, is a series of graphs depicting the binding of immune sera from guinea pigs (GP) immunized with monomeric gp120 YU2 (FIG. 6A; GP 60, 61), gp120 plus L5 (FIG. 6B; GP 62, 63) and gp120 plus HNG-156 (FIG. 6C; GP 64, 65) to peptides spanning the gp120 Group M Subtype B consensus sequence. Peptides are designated by the region in gp120 they belong to (e.g. C1) and the starting residue number (e.g. 133). Every fourth peptide is labeled. Peptides are 15 amino acids in length, with 11 residue overlap between adjacent peptides and were obtained from the NIH AIDS Research and Reference Reagent Program (catalog #9480). Some of the regions of relatively increased binding for [HIV-1_{YU2}gp120 +HNG-156] sera vs. other sera are highlighted: triangle, peptide 133-147 from the V1/V2 region; rectangle, peptides 313-335 from the V3 region; oval, peptide 353-367 from the C3 region.

### DETAILED DESCRIPTION OF THE INVENTION

The present invention is based on the discovery that specific, high affinity binding of an allosteric dual antagonist to HIV-1 gp120 induces a conformational change in the gp120 protein that traps gp120 in a three-dimensional structure that suppresses the protein's functionality and exposes novel antigenic epitopes present on the gp120 protein to immune surveillance by a host immune system. Inducing a conformational change in HIV-1 gp120and thereby exposing novel epitopes to host immune surveillance thereby elicits a novel, neutralizing immune response by the host immune system that neutralizes the binding of HIV-1 gp120 to host CD4 receptors. Accordingly, the present invention contemplates compositions and methods useful for the prevention and/or treatment of HIV-1 infection in a host subject.

### Definitions:

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

The techniques and procedures for recombinant manipulations, including nucleic acid and peptide synthesis, are generally performed according to conventional methods in the art and various general references (e.g., Sambrook et al, 2001, Molecular Cloning, A Laboratory Approach, Cold Spring Harbor Laboratory Press, Cold Spring Harbor, N.Y.; Ausubel et al., eds, 2005, Current Protocols in Molecular Biology, John Wiley & Sons, New York, N.Y.; and Gerhardt et al., eds., 1994, Methods for General and Molecular Bacteriology, American Society for Microbiology, Washington, DC), which are provided throughout this document.

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the term “heavy chain antibody” or “heavy chain antibodies” comprises immunoglobulin molecules derived from camelid species, either by immunization with a peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthetically or can be derived from a biological sample. Such a biological sample can include, but is not limited to a tissue sample, a tumor sample, a cell or a biological fluid.

The term “epitope” as used herein is defined as the part of a macromolecule that is recognized by the immune system, specifically antibodies, B cells, or T cells. An antigen can have one or more epitopes. Most epitopes recognized by antibodies or B cells can be thought of as three-dimensional surface features of an antigen molecule; these features fit precisely and thus bind to antibodies. Exceptions are linear epitopes, which are determined by the amino acid sequence (the primary structure) rather than by the 3D shape (tertiary structure) of a protein. In general, a B cell epitope is roughly about five amino acids and/or sugars in size. One skilled in the art understands that generally the overall three-dimensional structure, rather than the specific linear sequence of the molecule, is the main criterion of B-cell antigenic specificity and therefore distinguishes one epitope from another.

T cell epitopes are presented on the surface of an antigen-presenting cell, where they are bound to MHC molecules. T cell epitopes presented by MHC class I molecules are typically peptides between 8 and 11 amino acids in lengths, whereas MHC class II molecules present longer peptides, and non-classical MHC molecules also present non-peptidic epitopes such as glycolipids. Most antigens have many epitopes; i.e., they are multivalent.

As used herein “endogenous” refers to any material from or produced inside an organism, cell, tissue or system.

As used herein, the term “exogenous” refers to any material introduced from or produced outside an organism, cell, tissue or system.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

“Treating,” as used herein, means ameliorating the effects of, or delaying, halting or reversing the progress of a disease or disorder. The word encompasses reducing the severity of a symptom of a disease or disorder and/or the frequency of a symptom of a disease or disorder.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

The term “substantially pure” describes a compound, e.g., a protein or polypeptide, which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

By the term “applicator,” as the term is used herein, is meant any device including, but not limited to, a hypodermic syringe, a pipette, and the like, for administering the compounds and compositions of the invention.

“Instructional material,” as that term is used herein, includes a publication, a recording, a diagram, or any other medium of expression, which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Specifically bind” as used herein refers to the higher affinity of a binding

A “prophylactic” treatment is a treatment administered to a subject who does not exhibit signs of a disease or disorder or exhibits only early signs of the disease or disorder for the purpose of decreasing the risk of developing pathology associated with the disease or disorder.

A “therapeutic” treatment is a treatment administered to a subject who exhibits signs of pathology of a disease or disorder for the purpose of diminishing or eliminating those signs.

A “therapeutic agent” is any compound, composition, substance, or molecule that, when administered to a subject exhibiting signs of pathology of a disease or a disorder, produces a reduction of or eliminates those signs. As used herein, “therapeutically effective amount” refers to a nontoxic but sufficient amount of an agent to provide the desired biological result. The desired biological result in some instance can be a prophylactic and/or therapeutic treatment. That result can be reduction and/or alleviation of the signs, symptoms, or causes of a disease or disorder, or any other desired alteration of a biological system. An appropriate therapeutic amount in any individual case may be determined by one of ordinary skill in the art using routine experimentation.

“Pharmaceutically acceptable carrier” refers herein to a composition suitable for delivering an active pharmaceutical ingredient (API) to a subject without excessive toxicity or other complications while maintaining the biological activity of the API. Protein-stabilizing excipients, such as mannitol, sucrose, polysorbate-80 and phosphate buffers, are typically found in such carriers, although the carriers should not be construed as being limited only to these compounds.

As used herein, the term “physiologically acceptable” ester or salt means an ester or salt form of an active ingredient in a pharmaceutical composition which is compatible with any other ingredients of the pharmaceutical composition and which is not deleterious to the subject to which the composition is to be administered.

“Polypeptide” refers to a polymer composed of amino acid residues, related naturally-occurring, structural variants, and synthetic, non-naturally-occurring analogs thereof linked via peptide bonds. Synthetic polypeptides can be synthesized, for example, using an automated polypeptide synthesizer.

The term “protein” typically refers to long polypeptides. molecule for a target molecule compared to the binding molecule's affinity for non-target molecules. A binding molecule that specifically binds a target molecule does not substantially recognize or bind non-target molecules.

An “immunogen” comprises a compound, molecule, protein, or other agent that elicits an immune response in a subject when the immunogen is administered to the subject and contacts the cells of the subject's immune system. An immunogen of the present invention comprises an exogenous gp120 protein in combination with an allosteric dual antagonist.

As used herein, an “immunoassay” refers to any binding assay that uses an antibody capable of binding specifically to a target molecule to detect and quantify the target molecule.

As used herein, “conjugated peptide” refers to a peptide having one or more modified amino acids, such as γ-azidoproline, that introduce one or more functional groups useful for conjugation. The phrase also includes such modified peptides that have been conjugated to a compound.

As used herein, “metallocene” refers to an organometallic chemical compound with the general formula (C₅R₅)₂M consisting of two cyclopentadienyl rings bound on opposite sides of a central transition metal atom, M, and two cyclopentadienyl ligands coordinated in a sandwich structure, i.e., the two cyclopentadienyl anions are co-planar with equal bond lengths and strengths.

A “virus,” as used herein refers to a sub-microscopic infectious agent that is unable to grow or reproduce outside a host cell. Each viral particle, or virion, consists of genetic material such as DNA or RNA, within a protective protein coat called a capsid. The capsid shape varies from simple helical and icosahedral (polyhedral or near-spherical) forms, to more complex structures with tails or an envelope. Viruses infect cellular life forms and are grouped into animal, plant and bacterial types, according to the type of host infected.

A “host,” as used herein, refers to an individual, such an animal, preferably a human, that harbors a virus. In the case of HIV-1 infection, the host's immune system is targeted by the virus. antibody in the present invention is an antibody able to neutralize the interaction between HIV-1 gp120 protein and a host's CD4 receptor.

The phrase “neutralize the interaction between HIV-1 gp120 and a host's CD4 receptor,” as used herein, refers the ability of a substance, e.g. an antibody, to block, inhibit, reduce, or prevent the interaction of HIV-1 gp120 with a host CD4⁺ T cell receptor

The term “protect an animal against disease” is used herein to mean a reduction in the level of disease caused by HIV-1 in an animal inoculated with a composition comprising a dual antagonist of the invention and gp120, fragments, or variants thereof, compared with the level of disease caused by HIV-1 in an animal which has not been inoculated by a composition comprising a dual antagonist of the invention and gp120, fragments, or variants, thereof.

The term “virus neutralizing effective amount” as used herein means an amount of a substance, e.g. a protein, or antibody, which elicits an immune response when administered to a subject, which response is capable of neutralizing virus infectivity to a level which is less than 50% of normal infectivity in a standard virus neutralization assay, wherein normal infectivity is assessed relative to an otherwise identical animal to which the protein is not administered.

It is understood that any and all whole or partial integers between any ranges set forth herein are included herein.

### Description:

The present invention provides compositions and methods for neutralizing the interaction between HIV-1 gp120 and a host CD4 receptor. In one embodiment, a composition of the current invention comprises an allosteric dual antagonist. In another embodiment, a composition of the present invention comprises an allosteric dual antagonist without exogenous gp120. In still another embodiment, a composition of the present invention comprises an immunogen comprising a gp120 protein, peptide, or fragment thereof, in combination with an allosteric dual antagonist. An allosteric dual antagonist of the present invention is a molecule, protein, peptide, compound or composition that specifically, and with high affinity, binds the gp120 envelope protein of

The “immune system” of an organism, as used herein, refers to a collection of mechanisms, cells, tissues, organs, proteins, and molecules that protect the organism against disease by identifying, killing, and eliminating pathogens or cancerous cells. There are two key components to a vertebrate immune response to a pathogen or other immunologic challenge.

The “innate immune system” mounts an immediate, non-specific response comprising cell-mediated and humoral components. The innate immune response is largely triggered by a microbe or other pathogen identified by the host's immune system by pattern recognition receptors, which recognize components conserved among broad groups of microorganisms. The innate immune response includes inflammation, activation of the complement system, and the innate leukocytes including phagocytes (macrophages, meutrophils, and dendritic cells), mast cells, eosinophils, basophils, and natural killer cells.

“Adaptive immunity” evolved early in vertebrates and allows for stronger, more specific immunologic response as well as immunologic memory. The adaptive immune response is antigen-specific and requires the recognition of specific “non-self” antigens during a process called antigen presentation. Antigen specificity allows for the generation of responses that are tailored to specific pathogens or pathogen-infected cells. The ability to mount these tailored responses is maintained in the body by “memory cells.” Should a pathogen infect the body more than once, these specific memory cells are used to quickly eliminate it. B and T cells are specialized lymphocytes important in adaptive immunity. B cells are involved in the humoral immune response, whereas T cells are involved in cell-mediated immune response.

“Immune surveillance,” as used herein, refers to the idea that host immune cells circulate through the entire body of a vertebrate animal, including a human, and detect the presence of pathogens or genetically altered cells, mount an immune response to the pathogen or genetically altered cell, and eliminate it.

By the term “neutralizing antibody” as used herein, is meant an antibody that effects a reduction in the infectivity of a virus in the presence of the antibody compared with the infectivity of the virus in the absence of the antibody. A neutralizing HIV-1 and traps gp120 in a three dimensional conformation that either inhibits the interaction of gp120 with host cell surface receptors, exposes novel antigenic epitopes present on gp120 to host immune surveillance, or both.

An allosteric dual antagonist of the present invention may comprise a peptide triazole conjugate derived from the linear peptide 12p1 (RINNIPWSEAMM; SEQ ID NO. 1). In one embodiment, the allosteric dual antagonist is HNG-156. In another embodiment, the allosteric dual antagonist is a fragment of HNG-156. In still another embodiment, the allosteric antagonist is UM24, as shown in Formula 2, and the sequence of which is: Cit-N-N-I-X-W-S, where Cit is citrulline and X is an azidoproline conjugated to ethynylferrocene through the 3+2 cycloaddition reaction of alkynes and azides (SEQ ID NO. 8, as shown in Formula 2). In another embodiment, the allosteric dual antagonist comprises a recombinant “L5” cyanovirin-N-12p1 chimera.

An HIV-1 envelope gp120 protein of the present invention may be a monomer, a dimer, or a trimer of gp120. It may also be an analogue, variant, or a fragment of HIV-1 envelope gp120 protein. Accordingly, the components of the immunogen comprising an HIV-1 envelope gp120 protein and an allosteric dual antagonist may be combined as individual components of a composition. In one embodiment, the immunogen comprises a gp120 monomer bound non-covalently to an allosteric dual antagonist. In another embodiment, the immunogen comprises a gp120 trimer bound non-covalently to an allosteric dual antagonist. In another embodiment, the immunogen comprises a covalently cross-linked allosteric dual antagonist-gp120 fusion protein.

Thus, in one embodiment, the present invention comprises a composition comprising an allosteric dual antagonist which is administered to a subject in order to alter the conformation of an HIV-1 gp120 protein, and thereby suppress gp120 binding to receptors on a host cell and expose novel immunogenic epitopes present on the HIV-1 gp120 protein to host immune surveillance. In another embodiment, the present invention comprises a composition comprising an allosteric dual antagonist without exogenous HIV-1 gp120 which is administered to a subject in order to alter the conformation of an HIV-1 gp120 protein, and thereby suppress gp120 binding to receptors on a host cell and expose novel immunogenic epitopes present on the HIV-1 gp120 protein to host immune surveillance. In another embodiment, the present invention comprises a composition comprising an allosteric dual antagonist in combination with exogenous gp120 which is administered to a subject in order to alter the conformation of the exogenous HIV-1 gp120 protein, and thereby suppress gp120 binding to receptors on a host cell and expose novel immunogenic epitopes present on the HIV-1 gp120 protein to host immune surveillance.

In another embodiment, the present invention comprises a composition comprising an allosteric dual antagonist. In another embodiment, the present invention comprises a composition comprising an allosteric dual antagonist without exogenous HIV-1 gp120. In another embodiment, the invention comprises a composition comprising an allosteric dual antagonist in combination with exogenous gp120 which is administered to a subject in order elicit the production of novel, neutralizing antibodies that neutralize the interaction of HIV-1 gp120 and host cell surface receptors, such as the CD4 receptor.

The invention encompasses the preparation and use of a pharmaceutical composition comprising an allosteric dual antagonist. In another embodiment, the invention encompasses the preparation and use of a pharmaceutical composition comprising an allosteric dual antagonist without exogenous HIV-1 gp120. In another embodiment, the invention encompasses the preparation and use of a pharmaceutical composition comprising an immunogen of the invention. In one embodiment, the immunogen comprises an allosteric antagonist of HIV-1 gp120 in combination with exogenous HIV-1 gp120 envelope protein, a variant, or a fragment thereof, for administration to a subject in accordance with the present invention.

In another embodiment, the invention comprises the preparation and use of a pharmaceutical composition comprising HNG-156 for administration to a subject in accordance with the present invention. In another embodiment, the present invention comprises the preparation and use of a pharmaceutical composition comprising HNG-156 without exogenous HIV-1 gp120 for administration to a subject in accordance with the present invention. In another embodiment, the invention comprises the preparation and use of a pharmaceutical composition comprising of HNG-156 in combination with exogenous HIV-1 gp120 envelope protein, a variant, or a fragment thereof, for

In another embodiment, the present invention encompasses the preparation and use of a pharmaceutical composition comprising a neutralizing antibody generated in response to inoculating a subject with an immunogen of the invention, whereby the neutralizing antibody neutralizes the interaction of gp120 with a host cell receptor such as CD4.

In another embodiment, the present invention encompasses a pharmaceutical composition comprising a nucleic acid encoding a neutralizing antibody, whereby the neutralizing antibody neutralizes the interaction of gp120 with a host cell receptor such as CD4.

When an effective amount of a composition of the present invention is administered to a subject, the composition elicits a protective/neutralizing immune response, including antibodies that specifically bind to an antigenic epitope present on the HIV-1 gp120 protein, thereby preventing gp120 binding to or interacting with host immune cell receptors, preferably the CD4 receptor present on host T cells. Thus, a composition of the present invention can passively protect a subject from HIV infection or treat a host already infected with HIV-1.

A composition of the present invention is useful in the prevention of HIV-1 infection in a subject exposed to HIV-1 virus by any route likely to produce HIV-1 infection. In one embodiment, a subject is an animal, preferably a human. A composition of the present invention is also useful as a therapeutic agent for the treatment of ongoing infection by HIV-1. Thus the invention contemplates both prophylactic and therapeutic uses of the compositions of the invention.

The compositions and methods of the present invention may be practiced on any individual susceptible to or at risk for infection by HIV-1. In another embodiment, the composition and method of the invention may be practiced on any individual infected with HIV-1. Preferably, the individual is a non-human primate, more preferably, a human. A skilled artisan will appreciate that the compositions and methods of the present invention are not limited to the prevention and treatment of HIV-1, but also can be applied to the treatment and prevention of HIV-2.

It will be appreciated by a skilled artisan that the present invention may be used in combination with other therapies used to treat HIV-1 infection, including antiviral administration to a subject in accordance with the present invention.

In another embodiment, the invention comprises the preparation and use of a pharmaceutical composition comprising a fragment of HNG-156 for administration to a subject in accordance with the present invention. In another embodiment, the present invention comprises the preparation and use of a pharmaceutical composition comprising a fragment of HNG-156 without exogenous HIV-1 gp120 for administration to a subject in accordance with the present invention. In another embodiment, the invention comprises the preparation and use of a pharmaceutical composition comprising a fragment of HNG-156 in combination with exogenous HIV-1 gp120 envelope protein, a variant, or a fragment thereof, for administration to a subject in accordance with the present invention.

In another embodiment, the invention comprises the preparation and use of a pharmaceutical composition comprising UM24 for administration to a subject in accordance with the present invention. In another embodiment, the present invention comprises the preparation and use of a pharmaceutical composition comprising UM24 without exogenous HIV-1 gp120 for administration to a subject in accordance with the present invention. In another embodiment, the invention comprises the preparation and use of a pharmaceutical composition comprising of UM24 in combination with exogenous HIV-1 gp120 envelope protein, a variant, or a fragment thereof, for administration to a subject in accordance with the present invention.

In another embodiment, the invention comprises the preparation and use of a pharmaceutical composition comprising a recombinant “L5” cyanovirin-N-12p1 chimera for administration to a subject in accordance with the present invention. In another embodiment, the present invention comprises the preparation and use of a pharmaceutical composition comprising a recombinant “L5” cyanovirin-N-12p1 chimera without exogenous HIV-1 gp120 for administration to a subject in accordance with the present invention. In another embodiment, the invention encompasses the preparation and use of a pharmaceutical composition comprising a recombinant “L5” cyanovirin-N-12p1 chimera in combination with exogenous HIV-1 gp120 envelope protein, a variant, or a fragment thereof, for administration to a subject in accordance with the present invention. therapies, viral entry inhibitors (such as CCR5 co-receptor antagonists), fusion inhibitors, integrase inhibitors, nucleoside reverse transcriptase inhibitors, non-nucleoside reverse transcriptase inhibitors, protease inhibitors, and the like, including drugs used to treat opportunistic infections that result from HIV-1 infection.

### I. Compositions

### A. Allosteric Dual Antagonists

When the allosteric dual antagonist of the invention is a peptide triazole conjugate comprising the sequence INNIPWS (SEQ ID NO. 2) as the peptide component, the proline (residue 5 of SEQ ID NO. 1) is modified according to Formula I:

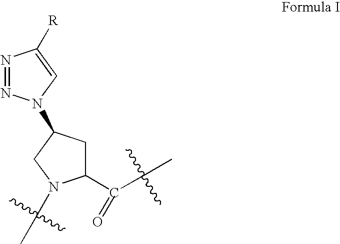

where R is a bulky aromatic group, excluding the R groups listed in Table 2 of Gopi et al., (2006, ChemMedChem 1:54-57). The excluded groups include phenyl, meta- and ortho-substituted phenyl, biphenyl, methyl-phenyl, ethyl-phenyl, 1-napthyl, 2-phenyl-ethyl. In an embodiment, R is selected from a naphthyl group, a para-alkyl-substituted phenyl, wherein the alkyl is methyl or ethyl, 2-phenylethyl and a metallocene. The naphthyl may be substituted or unsubstituted. Preferably, R is a metallocene. A metallocene is an organometallic chemical compound with the general formula (C₅R₅)₂M consisting of two cyclopentadienyl rings bound on opposite sides of a central transition metal atom, M. Exemplary transitional metals in the metallocene group are the 40 chemical elements 21 to 30, 39 to 48, 71 to 80, and 103 to 112 of the periodic table. In a preferred embodiment, the metallocene is ferrocene, Fe(C₅H₅)₂ (bis(η⁵-cyclopentadienyl)iron(II)).

Peptide triazole conjugates of the invention are antagonists of the binding reaction between HIV-1 envelope glycoprotein gp120 and CD4. Without wishing to be bound by any particular theory, it is believed that the peptide triazole conjugates of the invention are noncompetitive allosteric antagonists of the binding between gp120 and CD4. Antagonizing binding between gp120 and CD4 subsequently antagonizes the conformational change in the trimeric gp120 that allows gp120 to interact with a chemokine receptor (e.g., CCR5 or CXCR4). These steps lead to a fusion-active state that is crucial to the HIV infection process. Accordingly, the peptide triazole conjugates of the invention are useful for treating HIV-1 by reducing or precluding the fusion of HIV-1 viral particles to T-cells and thereby reducing or precluding HIV infection. Similarly, the peptide triazole conjugates are useful for reducing the risk of HIV infection in an individual at risk of HIV-1 exposure.

In preferred embodiments, the peptide triazole conjugates of the invention also inhibit binding of gp 120 and CCR5, or 17b. 17b is a monoclonal antibody that recognizes an epitope that overlaps the CCR5 binding site and is therefore considered in the art as a CCR5 surrogate. Inhibition of binding of 17b therefore is expected to correspond to inhibition of binding to CCR5. Accordingly, in preferred embodiments, the peptide triazole conjugates of the invention are dual antagonists.

In an embodiment, the peptide component of the peptide triazole conjugate of the invention consists essentially of SEQ ID NO. 1. In other embodiments, the peptide component comprising SEQ ID NO. 1 with the 1,2,3-triazol-modified proline as described herein comprises flanking residues on the N-terminus, the C-terminus, or both. Preferably, the peptide component comprises no more than about 50 residues, more preferably no more than about 30 residues, and more preferably still, no more than about 12 residues.

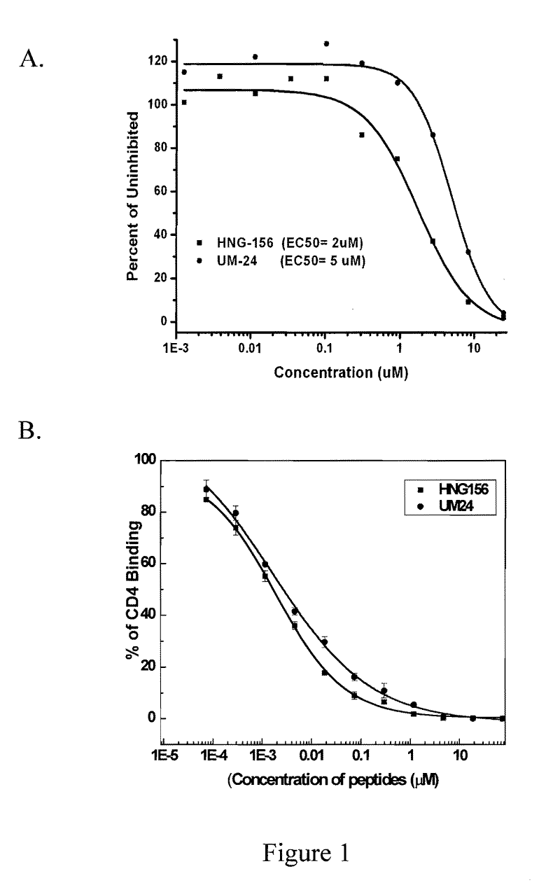

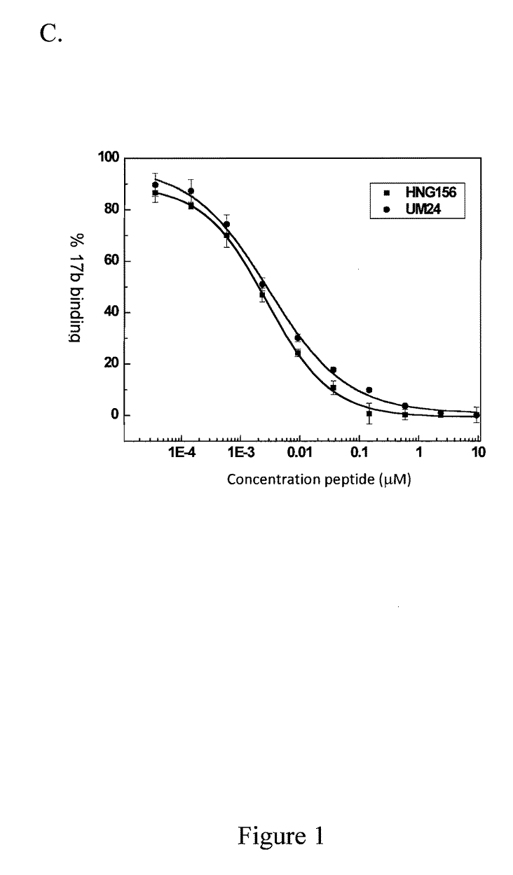

In an embodiment, the peptide triazole conjugate comprises RINNIPWSEAMM (“12p1”; SEQ ID NO. 3) as the peptide component, wherein proline 6 is replaced by a 4-substituted 1,2,3-triazole as described. R may be: a substituted or substituted naphthyl; a para-alkyl-substituted phenyl, wherein the alkyl is methyl or ethyl; 2-phenylethyl; or a metallocene. In one embodiment, R is an unsubstituted naphthyl and the peptide component consists essentially of SEQ ID NO. 3; this conjugate is referred to herein as “HNG-125”. In one embodiment, R is a para-alkyl-substituted phenyl, and the peptide component consists essentially of SEQ ID NO. 3. The conjugate wherein the alkyl group is a methyl is referred herein as “HNG-113”. The conjugate wherein the alkyl group is an ethyl is referred herein as “HNG-124”. The conjugate wherein R is 2-phenylethyl is referred herein as “HNG-137” (see FIG. 1B). In preferred embodiments, R is a metallocene and more preferably, is ferrocene. In one embodiment, the peptide component consists essentially of SEQ ID NO. 3. Preferably, in this embodiment, R is ferrocene; this conjugate is referred to herein as “HNG-156”.

HNG-156 has a high affinity (K_{d} about 7.4 nM as measured by SPR) for HIV-1 YU2 gp 120 envelope protein and similarly high affinity for gp 120 from two other HIV-1 strains. Furthermore, HNG-156 affinity has broad specificity for diverse subtypes and clades of HIV-1. HNG-156 has dual antagonism function, inhibiting gp120 binding to both host cell receptors (CD4 and CCR5). The inhibition exhibited is consistent with a non-competitive allosteric mode of action. The high affinity of HNG-156 for gp120 enables its use as part of a solid phase chromatographic medium useful for broad-specificity affinity chromatographic purification of HIV-1 or gp120 thereof, from diverse subtypes and clades of virus.

Like HNG-156, HNG-113, HNG-124, HNG-125 and HNG-137 also have dual antagonism function. The affinity of HNG-113 for HIV-1 YU2 gp120 is about 12 nM. The affinity of HNG-124 for HIV-1 YU2 gp120 is about 9 nM. The affinity of HNG-125 for HIV-1 YU2 gp120 is about 54 nM. The affinity of HNG-137 for HIV-1 YU2 gp120 is about 13 nM. The high affinities of these peptide triazole conjugates also supports their use in affinity purification of HIV-1 or gp120 therefrom.

UM24 is a seven amino acid peptide derived from HNG-156 as depicted in Formula 2: The sequence of UM24 (SEQ ID NO. 8) is Cit-N-N-I-X-W-S- where Cit denotes Citrulline and X denotes an azideoproline conjugated to ethynylferrocene through the 3+2 cycloaddition reaction of alkynes and azides (click chemistry) UM24 has an IC₅₀ of 2.6 μM for psuedoviral inhibition of HIV_{Bal} and inhibits CD4 and 17b binding to gp120_{YU2} with IC₅₀'s of 18 and 15 nM in molecular assays.

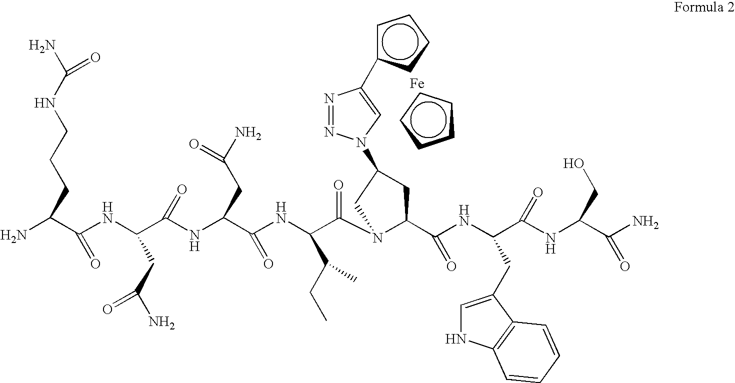

The invention also encompasses analogs and variants of the peptide triazole conjugates as well as analogies, variants, or fragments of gp120 of the invention. Analogs, variants, or fragments can differ from peptides by conservative amino acid sequence differences or by modifications which do not affect sequence, or by both.

For example, conservative amino acid changes may be made, which although they alter the primary sequence of the peptide, do not normally alter its function. Conservative amino acid substitutions typically include substitutions within the following groups:

glycine, alanine;

valine, isoleucine, leucine;

aspartic acid, glutamic acid;

asparagine, glutamine;

serine, threonine;

lysine, arginine;

phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine. Preferably, the modifications do not significantly impair the inhibition activity of the peptide triazole conjugate.

Information regarding the structure and function of the peptide component (e.g., SEQ ID NO. 1) is available to guide the skilled artisan in preparing peptide triazole conjugate analogs and derivatives useful in the methods of the present invention is available in the art. For instance, the Pro-Trp sequence has been identified as very important to the inhibition activity of the peptides (Ferrer et al, 1999, J Viol. 73:5795-5802). The effects of various substitutions and truncations of SEQ ID NO. 3 have also been studied (Bjorn et al., 2004, Biochem. 43:1928-1938). Thus, the skilled artisan has guidance to preparing analogs and derivatives which will retain inhibitory function.

Derivatives of the peptide component also include multiple triazoles at different positions of the peptide, for example, at both the proline and the tryptophan of the PW sequence.

The invention also encompasses peptide triazole conjugates, which have been modified using ordinary synthetic chemical techniques so as to improve their resistance to proteolytic degradation or to optimize solubility properties or to render them more suitable as a therapeutic agent. Analogs of such polypeptides include those containing residues other than naturally-occurring L-amino acids, e.g., D-amino acids or non-naturally occurring synthetic amino acids. The peptides of the invention are not limited to products of any of the specific exemplary processes listed herein. The peptides of the invention may further be conjugated to non-amino acid moieties that are useful in their therapeutic application. In particular, moieties that improve the stability, biological half-life, water solubility, and immunologic characteristics of the peptide are useful. A non-limiting example of such a moiety is polyethylene glycol (PEG).

Covalent attachment of biologically active compounds to water-soluble polymers is one method for alteration and control of biodistribution, pharmacokinetics, and often, toxicity for these compounds (Duncan et al., 1984, Adv. Polym. Sci. 57:53-101). Many water-soluble polymers have been used to achieve these effects, such as poly(sialic acid), dextran, poly(N-(2-hydroxypropyl)methacrylamide) (PHPMA), poly(N-vinylpyrrolidone) (PVP), poly(vinyl alcohol) (PVA), poly(ethylene glycol-co-propylene glycol), poly(N-acryloyl morpholine (PAcM), and poly(ethylene glycol) (PEG) (Powell, 1980, Polyethylene glycol. In R. L. Davidson (Ed.) Handbook of Water Soluble Gums and Resins. McGraw-Hill, New York, chapter 18). PEG possesses an ideal set of properties: very low toxicity (Pang, 1993, J. Am. Coll. Toxicol. 12: 429-456), excellent solubility in aqueous solution (Powell, supra), and low immunogenicity and antigenicity (Dreborg et al., 1990, Crit. Rev. Ther. Drug Carrier Syst. 6: 315-365). PEG-conjugated or “PEGylated” polypeptide therapeutics, containing single or multiple chains of polyethylene glycol on the polypeptide, have been described in the scientific literature (Clark et al., 1996, J. Biol. Chem. 271: 21969-21977; Hershfield, 1997, Biochemistry and immunology of poly(ethylene glycol)-modified adenosine deaminase (PEG-ADA). In J. M. Harris and S. Zalipsky (Eds) Poly(ethylene glycol): Chemistry and Biological Applications. American Chemical Society, Washington, D.C., p 145-154; Olson et al., 1997, Preparation and characterization of poly(ethylene glycol)ylated human growth hormone antagonist. In J. M. Harris and S. Zalipsky (Eds) Poly(ethylene glycol): Chemistry and Biological Applications. American Chemical Society, Washington, D.C., p 170-181).

It will be appreciated, of course, that the peptides may incorporate amino acid residues which are modified without affecting activity. For example, the termini may be derivatized to include blocking groups, i.e., chemical substituents suitable to protect and/or stabilize the N- and C-termini from “undesirable degradation,” a term meant to encompass any type of enzymatic, chemical or biochemical breakdown of the compound at its termini which is likely to affect the function of the compound, i.e., sequential degradation of the compound at a terminal end thereof

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal residue. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. De-carboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield de-amidated and de-carboxylated forms thereof without affect on peptide activity.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

As shown herein, peptides of the invention synergize strongly with cyanovirin-N in inhibiting HIV-1 infection. Accordingly, the invention provides a pharmaceutical composition comprising a peptide triazole conjugate of the invention and cyanovirin-N (CV-N), or a functional derivative thereof. CV-N binds to gp120 and inhibits HIV infection. An exemplary amino acid sequence for cyanovirin-N is SEQ ID NO. 4 (LGKFSQTCYNSAIQGSVLTSTCERTNGGYNTSSIDLNSVIENVDGSLKWQ PSNFIETCRNTQLAGSSELAAECKTRAQQFVSTKINLDDHIANIDGTLKYE). An exemplary coding sequence for cyanovirin-N is SEQ ID NO. 5 (CTTGTAAATTCTCCCAGACCTGCTACAACTCCGCTATCCAGGGTTCCGTTCTG ACCTCCACCTGCGAACGTACCAACGGTGGTTACAACACCTCCTCCATCGACCT GAACTCCGTTATCGAAAACGTTGACGGTTCCCTGAAATGGCAGCCGTCCAAC TTCATCGAAACCTGCCGTAACACCCAGCTGGCTGGTTCCTCCGAACTGGCTGC TGAATGCAAAACCCGTGCTCAGCAGTTCGTTTCCACCAAAATCAACCTGGAC GACCACATCGCTAACATCGACGGTACCCTGAAATACGAA).

In an embodiment, the peptide triazole conjugate is linked to cyanovirin-N. Linking may be either covalent or high affinity non-covalent linkage. Cyanovirin-N derivatives, such as a PEGylated CV-N, are useful in the invention as well. A PEGylated mutant CV-N, that retains anti-HIV activity has been reported (Zappe et al., 2008, Advanced Drug Delivery Reviews 60:79-87, Epub 16 August 2007).

Covalent attachments useful in linking a peptide triazole conjugate of the invention to cyanovirin-N include, but are not limited to, standard protein cross-linking chemistries, such as glutaraldehyde activation of amine-functionalized surfaces, trialkoxy aldehyde silanes, DMP (dimethyl pimelimidate), and N-hydroxysuccinimide active ester. Non-limiting examples of high affinity non-covalent attachments include hydrophobic interactions and avidin/biotin systems.

Linking a peptide triazole conjugate to cyanovirin-N may include peptide linkers, such as glycine rich linkers, such as Gly₄Ser. Multiples of this sequence may also be used to optimize the synergistic activity by altering the distance and rotational freedom between the two linked entities. Peptide linkers may be incorporated into the coding sequence for cyanovirin-N or may be included in the peptide synthesis of the peptide component of the peptide triazole conjugate.

Compounds useful in conjugating a molecule with biotin include, but are not limited to, aliphatic amines, carboxylic acid, DNP-X-biocytin-X, FMOC, hydrazide, iodoacetamide, maleimide, nitriloacetic acid and succinimidyl ester. Biotin, including various spacers, linking groups and the like, and methods of biotinylation are well known to the skilled artisan. See, for example, Savage et al., 1992, Avidin-Biotin Chemistry: A Handbook, Pierce Chemical Company, Rockford, Ill.; Diamandis et al., 1991, Clin. Chem. 37:625-636; DE 3629194; U.S. Pat. Nos. 4,709,037, 4,794,082, 4,798,795, 5,180,828, and 5,252,743; and WO 85/05638, each of which is incorporated herein by reference in its entirety.

Peptide coupling chemistry may be employed to link a peptide of the invention to cyanovirin-N directly or indirectly by means of a linking agent. The standard peptide coupling chemistry methods and procedures useful in this invention are readily available. Examples of books using these methods include, but are not limited to, the following citations incorporated herein by reference: P. D. Bailey, An Introduction to Peptide Chemistry, Ed.: John Wiley & Sons, 1990; Miklos Bodansky, Peptide Chemistry, A Practical Textbook, Ed.: Springer-Verlag, 1988; Miklos Bodansky, Principles of Peptide Synthesis, “Reactivity and Structure Concepts in Organic Chemistry,” Volume 16, Ed.: Springer-Verlag, 1984; and Miklos Bodansky, Principles of Peptide Synthesis, “Reactivity and Structure Concepts in Organic Chemistry,” Volume 21, Ed.: Springer- Verlag, 1984. See also U.S. Pat. Nos. 4,340,535 and 5,776,427 and EP 44167, each of which is incorporated herein by reference in its entirety.

A non-limiting example of preparing a peptide triazole conjugate of the invention linked to cyanovirin-N is as follows. A nucleic acid sequence encoding CV-N (e.g., SEQ ID NO. 5) is expressed in auxotrophic bacteria to contain a C-terminal linker with azidohomoalanine at the C-terminus. Optionally, the linker comprises one or more multiples of Gly₄Ser. The HNG-156 component is synthesized to contain an N-terminal propioloyl group to enable click chemistry conjugation. The azido group on the C-terminus of CV-N and the N-terminal alkyne group on HNG-156 will be reacted through copper-catalyzed 1,3-dipolar cycloaddition to form the triazole-linked chimeric fusion.

### B. Synthesis of Peptide Triazole Conjugate

The peptides of the invention are prepared using standard methods of in vitro peptide synthesis. Examples of solid phase peptide synthesis methods include the BOC method, which utilizes tert-butyloxcarbonyl as the α-amino protecting group, and the FMOC method, which utilizes 9-fluorenylmethyloxcarbonyl to protect the α-amino of the amino acid residues, both of which methods are well-known by those of skill in the art.

Exemplary methods for preparing γ-azidoproline, incorporating it into a peptide component, thereby forming a peptidyl azidoproline, and carrying out [3+2] cycloaddition with an appropriate alkyne to prepare of a peptide triazole conjugate of the invention are provided in the examples. The cycloaddition is carried out using click chemistry, which is well known in the art (Kolb et al., 2001, Angew. Chem. Int. Ed. 40:2004-2021). Appropriate alkynes to derivatize the azidoproline group with a specific bulky aromatic group, such as a naphthyl or a metallocene, are apparent to the skilled artisan.

Two routes for preparing a peptidyl azidoproline are provided, but the invention is not limited to these routes of preparation. A first route includes these steps:

1. Synthesis of γ-azidoproline.

2. Solid phase peptide synthesis using either Fmoc-chemistry or Boc-chemistry; γ-azidoproline is incorporated at the appropriate position during the solid phase synthesis.

3. Click chemistry (copper catalyzed 1,3 dipolar cycloaddition) is used to conjugate on solid phase using a naphthyl, an para-alkyl-substituted phenyl or metallocene alkyne (s) and the γ-azidoproline in the peptide component.

4. Cleave resulting peptide triazole conjugate from the solid support and purification using standard methods in the art, e.g., HPLC.

In one embodiment of this route, intermediate fragment coupling is used to couple the Fmoc-Ile-Azp-OH to the C-terminal fragment of the backbone on solid phase. For instance, Fmoc-Ile-Azp-OH is coupled to residue 7 of a fragment consisting of residues 7 through 12 of SEQ ID NO. 3, or to residue 6 of a fragment consisting of residues 6 and 7 of SEQ ID NO. 1. Synthesis of Fmoc-Ile-Azp-OH is described in the examples.

A second route for the synthesis of a peptidyl azidoproline comprises total solution phase synthesis, using fragment condensation. This route includes these steps:

1. Synthesis of γ-azidoproline

2. Peptide synthesis in solution phase using fragment coupling. Fragments of the peptide component with aziodoproline are synthesized using standard peptide chemistry with necessary protecting groups for side chain protection.

3. Click chemistry (copper catalyzed 1,3 dipolar cycloaddition) is used to conjugate on solid phase using a naphthyl, an para-alkyl-substituted phenyl or metallocene alkyne (s) and the γ-azidoproline in the peptide component.

4. Removal of protecting groups using standard protocols.

5. Purification of the resulting peptide triazole conjugate using standard methods

An alternative route to solution phase synthesis is to synthesize 4-substituted 1, 2, 3-1H- triazole-γ-substituted proline, using click chemistry conjugation, and use the conjugated proline in solution phase peptide synthesis. An exemplary method of preparing 4-substituted 1, 2, 3-1H-triazole-γ- substituted proline is provided in the examples. Use of this conjugated proline in solid phase peptide synthesis using Boc-chemistry or Fmoc-/Boc-strategy is also contemplated.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function, e.g. with DCC, can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

The resulting peptide triazole conjugate is purified, using standard peptide purification methods known in the art such as a solid phase matrix. Non-limiting examples of such methods include chromatographic methods including column chromatography, high pressure liquid chromatography (HPLC), and thin layer chromatography. Purification using an affinity column comprising an antibody that specifically binds to the peptide triazole conjugate is also useful. Confirmation of the peptide can be achieved using standard methods, including mass spectrometry techniques, such as MALDI-TOF.

### C. Immunological Compositions

The invention encompasses neutralizing antibodies generated by a subject that has been administered a composition comprising an immunogen of the invention. In one embodiment the invention encompasses neutralizing antibodies generated in a subject by administering a composition comprising a peptide triazole conjugate of the invention, such as HNG-156 and further comprising HIV-1 gp120, whereby the antibodies specifically bind to epitopes present on HIV-1 gp120, such that the binding of HIV-1 gp120 to host cell receptors, such as the CD4 receptor is prevented or blocked. In another embodiment the invention encompasses neutralizing antibodies generated in a subject by administering a composition comprising a peptide triazole conjugate of the invention, such as a fragment of HNG-156 and further comprising HIV-1 gp120, whereby the antibodies specifically bind to epitopes present on HIV-1 gp120, such that the binding of HIV-1 gp120 to host cell receptors, such as the CD4 receptor is prevented or blocked. In still another embodiment the invention encompasses neutralizing antibodies generated in a subject by administering a composition comprising a peptide triazole conjugate of the invention, such as UM24 and further comprising HIV-1 gp120, whereby the antibodies specifically bind to epitopes present on HIV-1 gp120, such that the binding of HIV-1 gp120 to host cell receptors, such as the CD4 receptor is prevented or blocked. In another embodiment, the present invention encompasses neutralizing antibodies generated in a subject by administering a composition comprising a recombinant L5 cyanovirin-N-12p1 chimera in combination with HIV-1 gp 120, whereby specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

Bacteriophage which encode the desired antibody may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv the antibodies specifically bind to epitopes present on HIV-1 gp120, such that the binding of HIV-1 gp120 to host cell receptors, such as the CD4 receptor, is prevented or blocked. Such antibodies may be polyclonal or monoclonal antibodies, or functional derivatives thereof.

The generation of polyclonal antibodies is accomplished by inoculating the desired animal with an immunogen of the invention and isolating antibodies which specifically bind the antigen therefrom.

Monoclonal antibodies directed against the immunogen may be prepared using any well known monoclonal antibody preparation procedures, such as those described, for example, in Harlow et al. (1998, In: Using Antibodies, A Laboratory Manual, Cold Spring Harbor, N.Y.) and in Tuszynski et al. (1988, Blood, 72:109-115). Human monoclonal antibodies may be prepared by the method described in U.S. patent publication 2003/0224490. Quantities of the desired immunogen may also be synthesized using chemical synthesis technology. Alternatively, DNA encoding the desired immunogen may be cloned and expressed from an appropriate promoter sequence in cells suitable for the generation of large quantities of immunogen. Monoclonal antibodies directed against the immunogen are generated from mice immunized with the immunogen using standard procedures as referenced herein.

Nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein. Further, the antibody of the invention may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA subject. In another embodiment, the method of the invention comprises administering a pharmaceutical composition comprising UM24 without exogenous gp120 to a subject to elicit an immunological response from a subject. In still another embodiment, the method of the invention comprises administering a pharmaceutical composition comprising a recombinant L5 cyanovirin-12p1 to a subject to elicit an immunological response from a subject. In still another embodiment, the method of the invention comprises administering a pharmaceutical composition comprising a recombinant L5 cyanovirin-12p1 without exogenous gp120 to a subject to elicit an immunological response from a subject. The method of the invention further comprises administering a neutralizing antibody to a host infected with HIV-1, thereby neutralizing the interaction of gp120 with host cell surface receptors such as CD4.

The method of the present invention comprises administering a pharmaceutical composition comprising an immunogen of the invention to a subject to elicit an immunological response from a subject. In another embodiment, the method of the present invention comprises administering a pharmaceutical composition comprising an allosteric dual antagonist in combination with gp120 to a subject to elicit an immunological response from a subject. In another embodiment, the method of the invention comprises administering a pharmaceutical composition comprising a peptide triazole conjugate in combination with gp120 to a subject to elicit an immunological response from a subject. In another embodiment, the method of the invention comprises administering a pharmaceutical composition comprising HNG 156 in combination with gp120 to a subject to elicit an immunological response from a subject. In another embodiment, the method of the invention comprises administering a pharmaceutical composition comprising a fragment of HNG 156 in combination with gp120 to a subject to elicit an immunological response from a subject. In yet another embodiment, the method of the invention comprises administering a pharmaceutical composition comprising UM24 in combination with gp120 to a subject to elicit an immunological response from a subject. In still another embodiment, the method of the invention comprises administering a pharmaceutical composition comprising a recombinant L5 cyanovirin-12p1 chimera in combination with gp120 to a subject to elicit an immunological response from a subject. The method of the invention further comprises fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995, J. Mol. Biol. 248:97-105).

### D. Small Molecules

When the allosteric dual antagonist is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are methods of making said libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

### II. Methods

The present invention encompasses a method of treating and preventing HIV-1 infection in an animal, preferably a human. In one embodiment, the present invention comprises a method of neutralizing the interaction of HIV-1 gp120 protein with a host cell surface receptor, such as a CD4 T cell receptor.

In one embodiment, the method of the present invention comprises administering a pharmaceutical composition comprising an allosteric dual antagonist to a subject to elicit an immunological response from a subject. In another embodiment, the method of the present invention comprises administering a pharmaceutical composition comprising an allosteric dual antagonist without exogenous gp120 to a subject to elicit an immunological response from a subject. In another embodiment, the method of the invention comprises administering a pharmaceutical composition comprising a peptide triazole conjugate to a subject to elicit an immunological response from a subject. In another embodiment, the method of the invention comprises administering a pharmaceutical composition comprising a peptide triazole conjugate without exogenous gp120 to a subject to elicit an immunological response from a subject. In another embodiment, the method of the invention comprises administering a pharmaceutical composition comprising HNG 156 to a subject to elicit an immunological response from a subject. In another embodiment, the method of the invention comprises administering a pharmaceutical composition comprising HNG 156 without exogenous gp120 to a subject to elicit an immunological response from a subject. In another embodiment, the method of the invention comprises administering a pharmaceutical composition comprising a fragment of HNG 156 to a subject to elicit an immunological response from a subject. In still another embodiment, the method of the invention comprises administering a pharmaceutical composition comprising a fragment HNG 156 without exogenous gp120 to a subject to elicit an immunological response from a subject. In yet another embodiment, the method of the invention comprises administering a pharmaceutical composition comprising UM24 to a subject to elicit an immunological response from a administering a neutralizing antibody to a host infected with HIV-1, thereby neutralizing the interaction of gp120 with host cell surface receptors such as CD4.

In another embodiment, the present invention comprises a method of exposing novel antigenic epitopes present on HIV-1 gp120 to host immune surveillance. The method comprises contacting HIV-1 gp120 with an allosteric dual antagonist, thereby forcing gp120 into a three dimensional conformation whereby novel antigenic epitopes present on gp120 are exposed to host immune surveillance, gp120 does not interact with host cell surface receptors such as CD4, or both. In one embodiment, the allosteric dual antagonist is a peptide triazole conjugate. In another embodiment the dual antagonist is a peptide triazole conjugate without exogenous go 120. In another embodiment the allosteric dual inhibitor is a peptide triazole conjugate in combination with gp120. In another embodiment, the allosteric dual inhibitor is HNG-156. In another embodiment, the allosteric dual inhibitor is HNG-156 without exogenous gp 120. In another embodiment, the allosteric dual inhibitor is HNG-156 in combination with gp120. In another embodiment, the allosteric dual inhibitor is a fragment of HNG-156. In another embodiment, the allosteric dual inhibitor is a fragment of HNG-156 without exogenous gp120. In another embodiment, the allosteric dual inhibitor is a fragment of HNG-156 in combination with gp120. In another embodiment, the allosteric dual inhibitor is UM24. In another embodiment, the allosteric dual inhibitor is UM24 without exogenous gp120. In another embodiment, the allosteric dual inhibitor is UM24 in combination with gp120. In another embodiment, the allosteric dual inhibitor is a recombinant “L5” cyanovirin-N-12p1 chimera. In another embodiment, the allosteric dual inhibitor is a recombinant “L5” cyanovirin-N-12p1 chimera without exogenous gp120. In another embodiment, the allosteric dual inhibitor is a recombinant “L5” cyanovirin-N-12p1 chimera in combination with exogenous gp120. In another embodiment, a composition comprising a neutralizing antibody generated in response to inoculation of a subject with a pharmaceutical compositions comprising a peptide triazole conjugate of the invention in combination with an HIV-1 gp120 protein or fragment thereof, can be used to neutralize the interaction of HIV-1 gp120 with a CD4 host cell receptor. In another embodiment, a composition comprising a nucleic acid encoding a neutralizing antibody generated in response to inoculation of a subject of a pharmaceutical compositions comprising a peptide triazole conjugate of the invention in combination with an HIV-1 gp120 protein or fragment thereof can be used to neutralize the interaction of HIV-1 gp120 with a CD4 host cell receptor.

An immunological response, as used herein, comprises a protective/neutralizing immune response mounted by a subject in response to inoculation by a specific antigen or combination of antigens. In the present invention, an immune response includes neutralizing antibodies that specifically bind to an antigenic site present on the HIV-1 gp120 protein, thereby preventing gp120 binding to or interaction with host immune cell receptors, preferably the CD4 receptor. Thus, a composition of the present invention can passively protect a subject from HIV infection.

The methods of the invention may be carried out with any individual susceptible to or at risk for infection by HIV-1. In another embodiment, the methods of the invention may be carried out with any individual infected with HIV-1. Preferably, the individual is a non-human primate, more preferably, a human.

The immunogen may be administered alone or in a pharmaceutical composition. The composition may further comprise other therapeutic agents. In a preferred embodiment, the composition further comprises CV-N.

In preferred embodiments, the methods of the invention, including the therapeutic and prophylactic methods, are practiced with a peptide triazole conjugate wherein the peptide component is SEQ ID NO. 1 and R is a metallocene. In preferred embodiments, the metallocene is ferrocene. In another preferred embodiment, the methods are practiced using a peptide triazole conjugate wherein the peptide component is SEQ ID NO. 3 and R is a metallocene. Preferably, the metallocene is ferrocene. In yet another embodiment, the therapeutic and prophylactic methods of the invention are practiced using a pharmaceutical composition comprising a peptide triazole conjugate of the invention and cyanovirin-N or a functional derivative thereof

### III. Pharmaceutical Compositions

The invention encompasses the preparation and use of pharmaceutical compositions comprising a peptide triazole conjugate of the invention. In another embodiment, the invention encompasses the preparation and use of pharmaceutical compositions comprising a peptide triazole conjugate of the invention without exogenous HIV-1 gp120. In another embodiment, the invention encompasses the preparation and use of pharmaceutical compositions comprising a peptide triazole conjugate of the invention and exogenous HIV-1 gp120, or a fragment thereof, for administration to a subject in accordance with the present invention.

In another embodiment, the present invention encompasses the use of pharmaceutical composition comprising an antibody generated in response to inoculation of a subject with a pharmaceutical compositions comprising a peptide triazole conjugate of the invention in combination with an HIV-1 gp-120 protein or fragment thereof.

In another embodiment, the present invention encompasses the use of pharmaceutical compositions comprising a nucleic acid encoding an antibody generated in response to inoculation of a subject of a pharmaceutical compositions comprising a peptide triazole conjugate of the invention in combination with an HIV-1 gp120 protein or fragment thereof.

Such pharmaceutical compositions may comprise the active ingredient alone, in a form suitable for administration to a subject, or the pharmaceutical composition may comprise the active ingredient and one or more pharmaceutically acceptable carriers, one or more additional (active and/or inactive) ingredients, or some combination of these.

The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Typically, dosages of peptide triazole conjugate, such as HNG-156, which may be administered to an animal, preferably a human, range in amount from about 1 μg to about 100 g per kilogram of body weight of the animal, and any and all whole or partial increments there between. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The pharmaceutical composition may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

Any route of administration is suitable for use in the therapeutic methods of the invention. Examples of routes of administration include oral, rectal, vaginal, parenteral, topical, pulmonary, intranasal, buccal, ophthalmic, intrathecal or another route of administration. Accordingly, pharmaceutical compositions that are useful in the methods of the invention may be prepared, packaged, or sold in formulations suitable for oral, rectal, vaginal, parenteral, topical, pulmonary, intranasal, intravenous, epidural, intraspinal, intra-arterial, buccal, ophthalmic, intrathecal, recombinant or another route of administration. Other contemplated formulations include projected nanoparticles, liposomal preparations, resealed erythrocytes containing the active ingredient, and immunologically-based formulations.

The invention encompasses the preparation and use of pharmaceutical compositions comprising a peptide triazole conjugate of the invention as an active ingredient that are useful for treatment of the diseases disclosed herein. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intracerebroventricular, surgical implant, internal surgical paint and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

### IV. Kits

The present invention includes a kit comprising an allosteric dual antagonist. In another embodiment, the present invention includes a kit comprising an allosteric dual antagonist without exogenous HIV-1 gp120. In another embodiment, the invention includes a kit comprising an immunogen of the invention where an immunogen of the invention comprises an allosteric dual antagonist of the invention in combination with an exogenous gp120 protein, peptide, or fragment thereof. An allosteric dual antagonist of the present invention, as defined elsewhere herein, is a molecule, protein, peptide, compound or composition that specifically, and with high affinity, binds the gp 120 envelope protein of HIV-1 and traps gp 120 in a three dimensional conformation that either inhibits the interaction of gp120 with host cell surface receptors, exposes novel antigenic epitopes present on gp120 to host immune surveillance, or both. Accordingly, in one embodiment a kit of the present invention may comprise a peptide triazole conjugate derived from the linear peptide 12p1 (RINNIPWSEAMM; SEQ ID NO: 1). In another embodiment, the allosteric dual antagonist is HNG-156. In another embodiment, the allosteric dual antagonist is a fragment of HNG-156. In another embodiment, the allosteric dual antagonist is UM24. In another embodiment, the allosteric dual antagonist comprises a recombinant “L5” cyanovirin-N-12p1 chimera.

A kit of the present invention may comprise a pharmaceutical composition comprising a composition of the invention. In one embodiment, a kit of the present invention may comprise a pharmaceutical composition comprising an allosteric dual antagonist. In another embodiment, a kit of the invention may comprise a pharmaceutical composition comprising an allosteric dual antagonist without exogenous HIV-1 gp120. In another embodiment, a kit of the invention may comprise a pharmaceutical composition comprising an immunological composition. A non-limiting example of an immunological composition useful in the practice of the invention includes neutralizing antibodies generated in a subject by administering a composition comprising a peptide triazole conjugate of the invention, such as HNG-156, or a fragment thereof such as UM24, and further comprising HIV-1 gp120, whereby the antibodies specifically bind to epitopes present on HIV-1 gp120, such that the binding of HIV-1 gp120 to host cell receptors, such as the CD4 receptor is prevented or blocked. In another embodiment, the present invention encompasses neutralizing antibodies generated in a subject by administering a composition comprising a recombinant L5 cyanivirn-N-12p1 chimera in combination with HIV-1 gp120, whereby the antibodies specifically bind to epitopes present on HIV-1 gp120, such that the binding of HIV-1 gp120 to host cell receptors, such as the CD4 receptor, is prevented or blocked. Such antibodies may be polyclonal or monoclonal antibodies, or functional derivatives thereof.

In still another embodiment, a kit of the invention encompasses the preparation and use of a pharmaceutical composition comprising an immunogen of the present invention, such as an allosteric dual antagonist in combination with a gp120 protein, peptide, or fragment thereof, or an immunological composition, such as a composition comprising neutralizing antibodies of the present invention.

A kit of the invention may provide a therapeutic agent in any form suitable for storage, including, but not limited to, a dry or lyophilized form. The kit may further provide the materials and means to reconstitute the therapeutic agent in a form compatible with the methods of the present invention, for example in a pharmaceutically acceptable carrier suitable for administration to a subject. A therapeutic agent of the present invention, including an allosteric dual antagonist either alone or in combination with exogenous HIV-1 gp120 protein, peptide, or fragment thereof, a neutralizing antibody, and the like may be packaged and stored separately in a kit, or provided as a combined composition.

A kit of the present invention further comprises instructional material which describes, for instance, administering a compound, composition, or therapeutic agent of the present invention to a subject as a prophylactic or therapeutic treatment as described elsewhere herein. In an embodiment, this kit further comprises a (preferably sterile) pharmaceutically acceptable carrier suitable for dissolving or suspending the therapeutic composition, comprising an immunogen or an immunologic composition of the invention, for instance, prior to administering the therapeutic agent to a subject. Optionally, the kit comprises an applicator for administering the therapeutic composition.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

1. Immunogens that Trap HIV-1 Env gp120 Protein in a Novel Conformational State: Monomers and Trimers

Immunogens include a gp120 monomer bound non-covalently to already-prepared high affinity peptide conjugate and chimera antagonists. HIV-1 Env gp120 protein with CD4 binding site small molecule ligand are also contemplated. Immunogens also include gp120 trimers and covalently cross-linked peptide conjugate-envelope protein fusion that will provide a stability-optimized immunogen.

a. Non-covalent gp120 and Trimer Complexes

Noncovalent complexes contain YU-2 gp120 monomer or trimer bound to already synthesized HNG peptides or CVN-12p1 chimeras. Immunogens comprising noncovalent gp120 monomer and trimer complexes are provided to Strategic Biosolutions for immunization of guinea pigs. Antisera obtained from the company are evaluated as described.

b. Cross-linked Conjugate-gp120 Fusion: Presenting the Allosteric State of HIV-1 Env Protein in a Single Stable Protein Immunogen

An azido-phenyl conjugated derivative of a dual antagonist peptide is cross-linked using ultraviolet (UV) radiation to monomeric gp120. The peptide or its derivatives are synthesized using click chemistry to covalently attach 4-ethynylaniline to azidoproline on the peptide backbone as described below. This will be followed by conversion of aryl amine to aryl azide and subsequent photolysis to form cross-linked conjugate-gp120 fusion. Prototype tests indicate that this process yields about multi-μg to mg amounts of cross-linked conjugate-gp120 needed for immunization. Covalent attachment of peptide to protein is confirmed by SDS-PAGE analysis followed by immunoblotting for the 6-His tag on the peptide. SPR data will be used to show that the 6-His tag on the N-terminus of the peptide (for detection and purification) does not lower the affinity of the peptide to gp120. The compound is isolated on a Nickel/Cobalt affinity column.

### Protein Reagents

HIV-1_{YU2}gp120 was produced as described previously in Drosophila S2 cells (Biorn et al., 2004, Biochem. 43:1928-1938; Pancera et al., 2005, J Virol. 79:9954-9969). Cells were spun down and supernatant sterile filtered. Supernatant was purified over an F105-antibody column (NHS-activated Sepharose, Amersham; F 105 antibody coupled according to manufacturer's instructions). HIV-1_{YU2} was eluted from the column with glycine buffer, pH 2.4, dialysed against PBS and frozen at −80° C. sCD4 was expressed in CHO cells in a hollow fiber bioreactor. Supernatant from the hollow fiber bioreactor was purified with an SP-column and bound fractions were then run over a Q-column. Unbound material was concentrated and analysed by SDS-PAGE. The gp120 proteins from HIV-1 SF162 and HIV-192UG037-08 were used in previous inhibitor binding studies (Cocklin et al., 2007, J Virol. 81:3645-3648); HIV-1SF162 gp120 was obtained through NIH AIDS Research and Reference Reagent Program from DAIDS and NIAID, while HIV-192UG037-08 gp120 was a gift as reported in Cocklin et al. (2007, J Virol. 81:3645-3648). The following monoclonal antibodies were obtained through the NIH AIDS Research and Reference Reagent Program: 2G12 from Dr Hermann Katinger; F105 from Dr Marshall Posner and Dr Lisa Cavacini; b12 from Dr Dennis Burton and Carlos Barbas.

### Synthesis of HNG-105 and HNG-156

HNG-105 was prepared as described in Gopi et al (2006, ChemMedChem 1:54-57). In brief, proline 6 of 12p1 (SEQ ID NO. 3) was replaced with (2S, 4S)-4-(4-phenyl-1H-1, 2, 3-triazol-1-yl) pyrrolidine-2-carboxylic acid. HNG-113, HNG-124, HNG-125 and HNG-137 were prepared as described in Gopi et al., (2008, J Med Chem. 51:2638-2647).

Materials used in the synthesis of HNG-156 are now described.

All Fmoc-protected amino acids, HBTU, HOBt and Hyp(OMe).HCl were purchased from Novabiochem. Fmoc-Rink amide resin was obtained from AppliedBiosystem. Solvents and other chemicals were purchased from Aldrich or Fisher and used without further purification. Peptides were synthesized on an automated peptide synthesizer (433A Applied Biosystem) at a 0.1 mmol scale. The peptides were cleaved from the resin by using a cocktail mixture of 95:2:2:1 trifluoroacetic acid/ethanedithiol/water/thioanisole. The crude peptides were purified by using C18 column on HPLC (Beckmann Coulter) with gradient between 95:5:0.1 and 5:95:0.1 water/acetonitrile/trifluoroacetic acid. The purified peptides were confirmed by MALDI-TOF.

Synthesis of Boc-Hyp(OMs)-OMe: Boc-L-trans-γ-hydroxyproline (2.45 g, 10 mmol) was dissolved in 50 mL of dry dichloromethane, cooled to 0° C., triethylamine (1.6 mL,12 mmol) was added followed by methanesulfonyl chloride (0.85 mL, 11 mmol). The reaction mixture was stirred at room temperature under N₂ for about 8 hours and diluted with 100 mL of dichloromethane. The reaction mixture was washed with 5% HCl, 5% Na₂CO₃ and water. After the evaporation of organic solvent, trans-4-mesyl derivative was separated as a solid (3.1 g, 96% yield) and used directly for the next step.

Boc-L-cis-4-azidoproline: Trans-4-mesyl proline derivative (1.61 g, 5 mmol) from the above step was dissolved in dry DMF. NaN3 (1.3 g, 20 mmol) was added. The reaction mixture was stirred overnight under N₂ at 70° C. The reaction mixture was poured into 50 mL of water and extracted with ethyl acetate (3×50 mL). The organic solvent was washed with water and dried over Na₂SO₄. After the evaporation of ethyl acetate under reduced pressure, methyl ester of Boc-L-cis-4-azidoproline separated as slightly yellowish oil (1.2 g, 90%). The methyl ester was subjected to base hydrolysis. Methyl ester of Boc-L-cis-4-azidoproline (1.08 g, 4 mmol) was dissolved in 10 mL of MeOH and 2 mL of 1N NaOH was added. The reaction mixture was stirred for 2 hours at room temperature and diluted with 50 mL of water. The MeOH was removed under reduced pressure and the aqueous layer extracted with ether (3×20 mL). The aqueous layer was acidified to pH 3 by using % 5 HCl and extracted with ethyl acetate (3×50 mL). After the aqueous work-up, the organic solvent was evaporated under reduced pressure to yield 0.97 g (95%) of Boc-L-cis-4-azidoproline.

Fmoc-L-cis-4-azidoproline: Boc-L-cis-4-azidoproline (0.76 g, 3 mmol) was dissolved in 5 mL of dichloromethane, cooled to 0° C. 5 mL of trifluoroacetic acid was added and stirred for 30 minutes. The solvent was evaporated, the residue was dissolved in 50 mL of water and the pH was adjusted 8 by adding solid Na₂CO₃. Fmoc-OSu (1 g, 3 mmol) was dissolved in 10 mL of THF and added to the reaction mixture. After completion of the reaction, the reaction mixture was extracted with ether (3×50 mL). The aqueous layer was acidified to pH 2. The separated white precipitate was extracted with ethyl acetate (3×50 mL). After the aqueous work-up and evaporation of organic solvent, the yield was 0.98 g (87%) of Fmoc-L-cis-4-azidoproline. A pure sample was obtained after recrystallization from ethyl acetate/n-heptane. The pure Fmoc-L-cis-γ-azidoproline was directly used in peptide synthesis.

HNG-156 (SEQ ID NO. 6; RINNIXWSEAMM, where residue 6 is (2s, 4s)-4-(4-ferrocenyl-1H-1, 2, 3-triazol-1-yl) pyrrolidine-2-carboxylic acid) was prepared by two different routes.

### Route I to HNG-156 Synthesis: Solid Phase Synthesis of HNG-156 Utilizing Intermediate Fragment Coupling of Fmoc-Ile-Azp-OH to [7-12] Fragment on Solid Phase

### Fragment Coupling Strategy for Synthesis of HNG-156 on a Solid Phase

Peptide RINNI(Azp)WESAMM (SEQ ID NO. 7, wherein residue 6 is azidoproline) was synthesized on the solid support. In the process of continuous synthesis, it was found that the coupling between azido proline 6 to isoleucine 5, leads to incomplete coupling. To address this problem, triple coupling of isoleucine 5 was used and finally the unreacted imine group of azido proline was blocked with acetic anhydride to avoid the interference of isoleucine-deleted peptide in the purification of final peptide.

In this process, the final yield of the peptide was less than expected. To address this problem, a fragment coupling strategy in solid phase peptide synthesis was utilized. Fmoc-Ile-Azp-OH was synthesized in the solution phase and coupled to free amine of Trp7 on the resin.

Synthesis of Fmoc-Ile-Azp-OH in solution phase. Succinimidyl active ester of Fmoc-Ile (4.5 g, 10 mmols) was dissolved in 50 mL of DMF. Unprotected γ-azidoproline (1.87g, 12 mmols) was dissolved in 25 mL of 20% Na₂CO₃ solution and added to succinimidyl ester of Fmoc-Ile. The reaction was stirred for about 12 hrs at room temperature. The reaction mixture was poured into 100 mL of water and extracted with ether (3×30 mL) to remove the unreacted Fmoc-Ile-OSu. The aqueous phase was acidified to pH 3 using 10% HCl. The liberated Fmoc-dipeptide acid was extracted to ethyl acetate (3×50 mL). The combined ethyl acetate was washed with 5% HCl, water, brine solution and passed over anhydrous Na₂SO₄. After the evaporation of ethyl acetate, the crude product was recrystallized using ethyl acetate and hexane. Overall yield was 4.2 g (83%). The Fmoc-dipeptide acid was directly used in the solid phase synthesis without further purification.

[3+2] Cycloaddition reaction on resin: The resin of protected peptide (0.1 mmol), with L-cis-4-azidoproline group, was suspended in 5 mL of acetonitrile/water/DIEA/pyridine (4:4:1:0.5) mixture. The terminal alkyne ethynylferrocene (0.21 g, 1 mmol) was added, followed by a catalytic amount of Cu(I). The reaction was stirred overnight at room temperature over night; the solution was filtered and washed with 5% HCl, an excess of DMF and dichloromethane. HNG-156 was cleaved from the resin by using a cocktail mixture of 95:2:2:1 trifluoroacetic acid/ethanedithiol/water/thioanisole and purified by HPLC using a C-18 column. The peptide was confirmed by MALDI-TOF.

### Route II to HNG-156: Total Solution Phase Synthesis

HNG-peptide conjugates were also synthesized by conventional solution-phase methods, using a fragment condensation strategy (Bodanszky, M.; Bodanszky, A. The Practice of Peptide Synthesis, 2nd. ed. Springer-Verlag, New York, 1994). The t-butyloxycarbonyl group was used as N-terminus protection, while the C-terminus was protected as a methyl ester. Intermediate deprotections were performed with 50% trifluoroacetic acid in dichloromethane and saponification (1 N NaOH and methanol) for the N- and C-termini, respectively. Couplings were mediated by dicyclohexylcarbodiimide (DCC)/1-hydroxybenzotriazole (HOBt). All the coupling reactions were monitored by using TLC (thin layer chromatography). The intermediate peptides were purified by column chromatography. γ-Azidoproline (Azp) was synthesized using methyl ester of hydroxyproline (Hyp-OMe) (Gopi et al., (2006) ChemMedChem 1: 54-7).

The following peptides are sub-sequences of HNG-156 (SEQ ID NO. 6).

The N-terminus dipeptide acid Boc-Arg(Boc)2-Ile-OH was prepared by Boc-Arg (Boc)2-OSu (succinimidyl active ester). The tetrapeptide Boc-Arg (Boc)2-Ile-Asn-Asn-Ome was prepared by [2+2] condensation, involving Boc-Arg(Boc)2-Ile-OH and H-Asn-Asn-OMe.

The pentapeptide Boc-Ser(OBzl)-Glu(Bzl)-Ala-Met-Met-OMe was prepared by [2+3] condensation involving an N-terminus dipeptide acid Boc-Ser(OBzl)-Glu(Bzl)-OH and C-terminus deprotected tripeptide H-Ala-Met-Met-OMe using DCC/HOBt. The octapeptide Boc-Ile-Azp-Trp-Ser(OBzl)-Glu(Bzl)-Ala-Met-Met-OMe was prepared by [3+5] coupling involving Boc-Ile-Azp-Trp-OH and H-Ser(OBzl)-Glu(Bzl)-Ala-Met-Met-OMe.

At the final step, the tetrapeptide acid (Boc-Arg(Boc)2-Ile-Asn-Asn-OH) was coupled to the N-terminus deprotected octapeptide (H-Ile-Azp-Trp-Ser(OBzl)-Glu(Bzl)-Ala-Met-Met-OMe). The resulting peptidyl azidoproline (peptide with γ-azidoproline) was purified using column chromatography.

The peptidyl azidoproline was subjected to click conjugation at a preparative scale as described in the literature (Kolb et al., 2001, Angew. Chem. Int. Ed. 40: 2004-2021). The peptide was dissolved in 1:1 tert-butanol/water, ethynylferrocene was added followed by 5 mol % of CuSO₄.5H₂O and sodium ascorbate. The final peptide was subjected to hydrozenolysis using Pd/C in methanol for the removal of benzyl groups. Finally the Boc-groups were removed by using 2 M HCl in dioxane. The final peptide triazole conjugate was purified using preparative HPLC.

Alternatively, 4-substituted 1, 2, 3-1H- triazole-γ-substituted proline was synthesized in solution starting from methyl ester of Boc-protected-cis-γ-substituted proline using the above described protocol. After the click conjugation, the product was extracted into ethyl acetate. The click conjugated proline methyl ester was purified by column chromatography using ethyl acetate/hexane (35/65) solvent mixture. The purified product was subjected to saponification. The click conjugated Boc-protected proline was used in the above described solution phase peptide synthesis. This product may also useful in the Boc-chemistry based solid phase peptide synthesis. Further, to obtain Fmoc-protected 4-substituted-1, 2, 3-1H- triazolyl-proline derivative, which is useful for solid phase peptide synthesis in Fmoc-/Boc-strategy, the Boc-group of the click conjugated proline was deprotected and protected again with an Fmoc-group using Fmoc-OSu. The final product was isolated and used in the peptide synthesis.

UM24 is a seven amino acid peptide derived from HNG-156 as depicted in Formula 2. The sequence of UM24 (SEQ ID NO. 8) is Cit-N-N-I-X-W-S- where Cit denotes Cutrulline and X denotes an azideoproline conjugated to ethynylferrocene through the 3+2 cycloaddition reaction of alkynes and azides.

### Production of Guinea Pig Antisera

### Guinea Pig Antisera and Screening for Neutralization Activity

Guinea pigs are immunized with 0.2 mg immunogen per injection (Strategic Biosolutions; Newark, Del.). Four subsequent immunizations are given at 2 week intervals and antisera collected (Strategic Biosolutions, Newark, Del.). Neutralizing activities of sera/IgG are tested using ELISA binding and syncytium formation assays. Sera/IgG are also tested for inhibition of viral infection of cells using a pseudoviral inhibition assay (Biorn, et al., 2004, Biochemistry 43:1928). Sera prioritized based on results of these initial assays are tested for breadth of neutralizing activity against multiple clades and subtypes of HIV-1 using pseudoviral inhibition assays.

### Profiling Antibody Response by Peptide Library ELISA

For mapping of the epitopes of the polyclonal antibodies generated within this proposal, a set of overlapping gp120 peptides based upon the HIV-1 clade B Env consensus sequence conB (AIDS Research and Reference Reagent Program) is used. These peptides are 15 amino acids in length with 11-amino acid overlaps between sequential peptides. A randomized 15-mer peptide, such as from gp41, is synthesized in-house and used as a control for background subtraction. Peptides from the library are immobilized on ELISA plates, blocked, washed and then incubated with whole sera or IgG-containing fractions from control and immunized guinea pigs. Antibody binding are detected with anti-guinea pig HRP-secondary antibody (Wang, et al., 2002, Vaccine 21:89; Shen etal., 1999, Vaccine 17:3039).

### Pseudovirus Production and Infection for UM24 Characterization

Envelope-pseudotyped, luciferase-reporter viruses were produced utilizing envelope expression vectors co-transfected into 293T cells using calcium phosphate precipitation (Profection Mammalian Transfection System, Promega) or Fugene reagent (Qiagen) together with the envelope-deficient pNL4-3-Fluc+env−provirus developed by N. Landau {Connor, 1995 #210}. Culture supernatants containing pseudotyped particles were collected 48-72 hours after transfection, clarified by centrifugation, aliquoted and stored at minus 80° C. until use. At 48-72 hours post-infection, these cells were washed with PBS and lysed. The amount of entry mediated by each envelope was measured by detecting luciferase activity (Luciferase Assay System, Promega) with a microplate luminometer. For inhibition experiments, the pseudotype stocks were first incubated at 37° C. for 30-45 minutes. IC₅₀ values were estimated using non-linear regression analysis with Origen 7 (Northampton, Mass.). All experiments were performed at least in duplicate and results were expressed as relative infection with respect to untreated (100%). Result: HNG-156 and UM24 neutralized pseudovirus with IC50's of 0.9 and 2.6 μM, respectively.

### Immunizations and Pseudoviral Neutralization by Antisera

SBF pathogen free rabbits (4 rabbits per immunogen) were injected with 200 μg of the following gp120_{YU2}, gp120_{YU2}/HNG-156 non-covalent mixture (1:4 protein:peptide molar ratio), gp140-FOLDON (trimeric envelope protein), gp140_{YU2}-FOLDON/HNG-156 non-covalent mixture (1:4 molar ratio). All immunogens were formulated in Freund's incomplete adjuvant (Sigma) except for the first injection which was in Freund's complete adjuvant.

Bleeds were taken 10 days post each immunization, except for animals 97-00 6th bleed which was taken 19 days after the 6^{th} immunization.

Prebleed (the bleed before the 1^{st} injection of immunogen) and end-bleeds (Bleed 6) were assayed for inhibition of pseudotyped HIV in a Tzm-bl assay where the target cells express CD4 and the coreceptors CCR5 and CXCR4 and also a Tat-inducible luciferase gene whose expression is dependent on the Tat protein expressed by the viral genome. Thus, light readout upon addition of D-luciferin is directly correlated with the amount of infection that has taken place. The serum dilution resulting in 50% reduction in infection is presented.

The purpose of the present experiments was to examine the extent to which the HIV-1 envelope structure induced by the allosteric dual antagonists could elicit novel and neutralizing antibodies when used to inoculate a subject. The results of the experiments presented in this Example are now described.

### Experimental Example 1

### Antigenic Mapping of gp120 Using HNG-156 or UM-24

### A. Characterization of UM24

UM24 has 2.6 μM IC₅₀ for psedudoviral inhibition of HIV_{BaL} (FIG. 1A). UM24 inhibits CD4 binding to gp 120 with an IC₅₀ of 18 nM in molecular ELISA assays (FIG. 1B). LTM24 inhibits 17b binding to gp120 with an IC₅₀ of 15 nM in molecular ELISA assays (FIG. 1C).

B. Antibody Direct Binding to gp120

Monoclonal antibodies (culture supernatant or purified IgG) were incubated at varying concentrations with adsorbed gp120 of clade YU2, BaL or Hxbc2 (depending on mAb reactivity)in ELISA format. Bound mAb was detected with species specific secondary antibody-HRP conjugate and an OPD assay at 450 nm. Result: All antibodies bound at least one of the gp120 clades tested: YU2, BaL or Hxbc2. An antibody or concentration or culture supernatant dilution that gave maximum signal and was sub-saturating was chosen for subsequent competition analyses.

b12 binding to gp120_{YU2} was assayed by SPR using a Biacore 3000 instrument. 1100 RU of mAb b12 was immobilized by NHS coupling on a CM5 surface and unreacted sites were blocked by injection of 1 M ethanolamine. gp120 purified via its His₆ tag on a Nickel column was injected at 100 μl/min with 2.5 min. association and 2 min. dissociation time on b12 and control (mAb 2E3) surfaces. Buffer (PBS+0.005% Tween-20) injection and control surface responses were subtracted from the injection of gp120 over b12 and binding sensorgrams were obtained.

A concentration of gp 120 (400 nM) that resulted in maximum response without saturating the surface was chosen for subsequent competition assays.

### C. CD4 Binding to Hxbc2 and BaLgp120

100 ng gp120 was adsorbed to ELISA plates and sCD4 at varying concentrations was added in triplicates. Bound sCD4 was detected with biotin-anti-CD4 (OKT4) and Streptavidin-HRP, followed by reading of OPD reaction at 450 nm.

A concentration of sCD4 that gave maximum signal without saturating the surface was chosen for subsequent use of CD4 as a positive control for competition by UM24 on Hxbc2 and BaL gp 120 surfaces (which did not bind 17b or bound the anti-human antibody used to detect 17b non-specifically, respectively). The following concentrations of CD4 were chosen: for Hxbc2: 3.1nM, for BaL: 7 nM.

D. UM24 and HNG-156 Inhibition of gp120 binding to CD4 and 17b

Inhibition of gp120_{YU2} binding to CD4 and 17b: 100 ng gp120_{YU2} was adsorbed to ELISA wells and unreacted sites in the wells were blocked with 3% BSA in PBS. 2 nM mAb 17b or 10 nM CD4 (final concentration) were mixed with UM24 or HNG156 of varying concentrations, added to the wells in triplicates and incubated at room temperature (RT) for 1 hr. After washing, bound 17b was detected with anti-human-HRP conjugated secondary antibody (Chemicon, Australia) and OPD assay at 450 nm. Bound sCD4 was detected with biotinylated anti-CD4 antibody (OKT4, eBiosciences) and streptavidin-HRP (Anaspec), followed by OPD assay at 450 nm. Result: UM24 and HNG156 were found to inhibit CD4 and 17b binding with IC50's ranging from 15 to 18 nM.

Inhibition of gp120_{Hxbc2} and gp120_{BaL} binding to CD4: 100 ng sCD4 was adsorbed per well and blocking was done with 3% BSA. gp120_{Hxbc2} and gp120_{BaL} were prepared at varying concentrations in 0.5% BSA and added to the wells in triplicates. After 1 hr. incubation at RT, wells were washed with PSB +0.1% Tween-20 three times and bound gp120 was detected with D7424 anti-gp120 antibody (Aalto Biosciences) and anti-sheep-HRP secondary antibody, followed by OPD assay at 450 nm.

UM24 inhibited CD4 binding to gp120_{BaL} and gp120_{Hxbc2} with IC₅₀'s of 21 and 15 nM, respectively.

E. Antibody Competition with UM24

100 ng gp120_{YU2} was adsorbed to wells. 0.5% BSA with and without UM24 (final concentration=1000, 100 or 10 uM) was added to the wells, followed immediately by addition of the antibody. Bound antibody was detected with species specific secondary antibody −HRP, and read with OPD assay at 450 nm. 48d inhibition was determined using adsorbed gp120 (100 ng/well) in ELISA format. Culture supernatant containing 48d was added to the wells at serial dilutions starting at 1:20, with and without HNG-156 (2 μM final concentration). Bound 48d was detected with anti-human HRP and OPD assay 450 nm. 2 nM 17b was also tested for inhibition by HNG-156 at the same time using identical methods, and showed 1.2% binding in the presence of HNG-156.

b12 and m18 competition with HNG-156 and UM24, were measured by surface plasmon resonance (SPR; Biacore 3000, GE Healthcare) where the antibodies were immobilized by NHS coupling on a CM5 surface. 100 or 400 nM gp120_{YU2}was mixed with increasing concentrations of HNG-156 or UM24 for the m18 and b12 assays, respectively and injected over the antibody surface. m18 was injected at 50 μl/min with 200 sec. association and 300 sec. dissociation. Inhibition curves were generated from response at the end of association. b12 was injected at 100 ul/min with 2.5 min. association and 2 min. dissociation and binding curves were generated from responses at the 100^{th} second of dissociation.

### F. Binding of Linear and Conformational Antibodies to gp 120

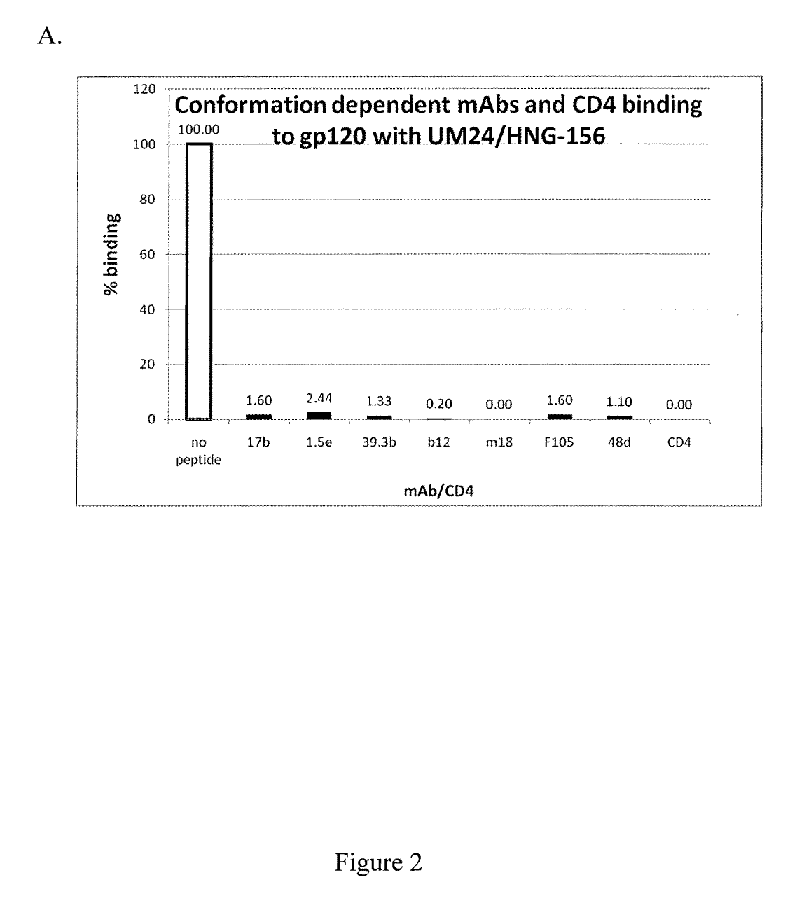

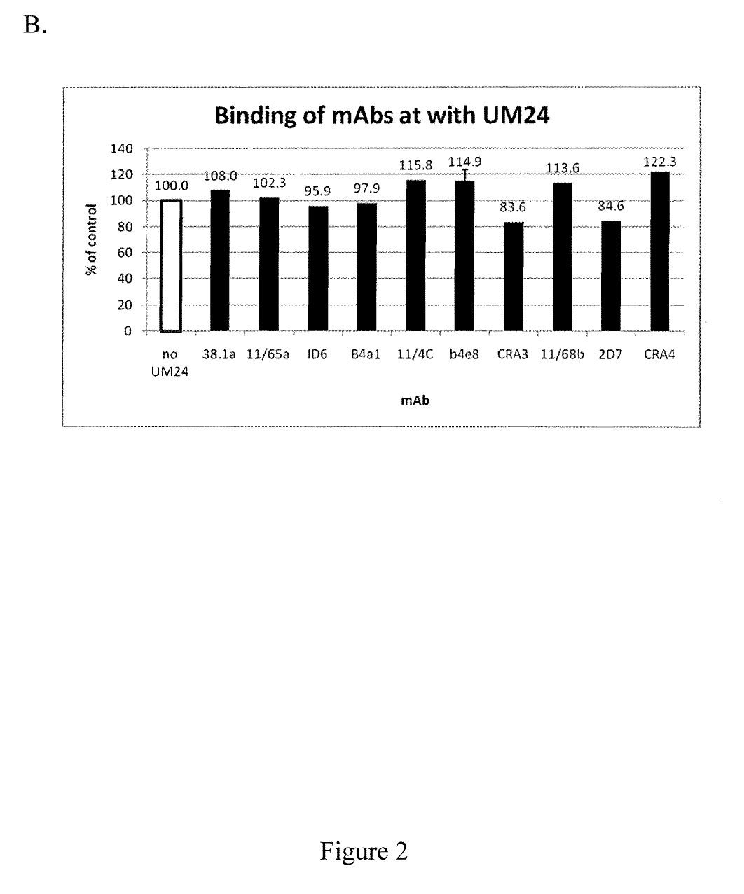

ELISA and SPR competition between UM24/HNG-156 and conformation-sensitive ligands (FIG. 2A). 100 ng gp120_{YU2} was adsorbed to wells. 0.5% BSA with and without UM24 (final concentration=100 uM) was added to the wells, followed immediately by addition of the antibody. Bound antibody was detected with species specific secondary antibody-HRP, and read with OPD assay at 450 nm. Assays were done at least twice in triplicates with similar results. One set is shown. 48 d inhibition was determined using 2 μM HNG-156. M18 and F105 inhibition were determined using 1 μM and 0.6 μM HNG-156, respectively, using surface plasmon resonance (SPR; Biacore 3000, GE Healthcare) as described in methods.

Binding of linear epitope antibodies to gp120 in the presence of 1 mM (11/4C), 10 uM (38.1a, 11/65a, ID6, F425 B4a1) or 100 uM (F425 b4e8, CRA3, 11/68b, 2D7 and CRA4) UM24 (FIG. 2B). Assays were done In ELISA format as explained in methods for the conformation-sensitive antibodies. Binding values were normalized to those obtained without UM24 (unfilled bar). All assays were done once in triplicates except for b4e8 which is an average of two experiments. CRA4 inhibition was repeated once with 10 uM UM24 with similar results.

### G. Epitope Mapping

### 1. Conformation Antibodies:

The CD4 binding site on gp120 comprises amino acids 97, 124, 126, 129, 195, 196, 198, 257, 279, 280-3, 365-8, 370-1, 425-32, 455-61, 469, 472-7, and 480. CD4 binding was inhibited to <5% by 400 nM UM24 on gp120. 100 ng CD4 was adsorbed to ELISA plate and UM24-gp120 mixture containing varying amounts of UM24 and 10 nM gp120_{YU2} were added in triplicates. Bound gp120 was detected with D7324 anti-gp120 antibody. Assay was done twice. Assays with Hxbc2 and BaL gp120 and 100 μM UM24 gave similar results. PDB ID is 2qad for all mAb epitope diagrams except 2D7.

The binding site for conformational antibody mAb 39.3b (Cordell et al., 1991, Virology 185:72-79) comprises amino acids 88, 113, 257, 368, 370, 384, 482-4 of gp120. 0.5% BSA with and without UM24 were added to gp120_{YU2} adsorbed to ELISA wells, followed immediately by addition of 39.3b. Final concentration of UM24 was 100 μM. After 1 hr incubation at RT and washing, bound mAb was detected with anti-human-HRP secondary antibody and OPD reading at 450 nm. 39.3b was inhibited to 1.5% (of the 0.5% BSA control) by UM24. Other mAb competition experiments were done similarly, except in some cases where 10 uM UM24 was used as noted, and for b12 which was analyzed by SPR.

The binding site for conformational antibody mAb b12 (Zhou et al., 2007, Nature, 445: 732) comprises amino acids 257, 280-2, 364-375, 384, 386, 417-9, 453, 455-6, 472-5 of gp120.

b12 competition with UM24 was assayed by SPR (Biacore 3000) where varying concentrations of UM24 mixed with 400 nM gp120_{YU2} were passed over a b12 surface at 100 μl/min with 2 min association and 1 min. dissociation. UM24 concentration ranged from 100 μM to 98 nM. b12 was inhibited to completion (0% of control) by 25 μM UM24.

The binding site for conformational antibody mAb F105 comprises amino acids 257, 368, 370, 375, 383, 421, 457, 470, 474, 480 of gp120. F105 competition by HNG-156 was assayed as described in Gopi, 2009, J. Mol. Recognition 22:169-174). F105 was inhibited to near completion at the concentrations tested.

The binding site for conformational antibody mAb m18 (Prabakaran et al., 2006, J. Mol. Biol. 357: 82-99) comprises amino acids C119, S199, K207, T257, N339, P363, G367, D368, P369, E370, Y384, N386, N392, K421, E429, R469, P470, G472, D474, M475, W479 of gp120. m18 competition with HNG-156 was assayed by SPR using immobilized m18 and 100 nM gp120_{YU2} as analyte. m18 binding to gp120 was inhibited to completion (0%) by 1 uM HNG-156.

The binding site for conformational antibody mAb 48d comprises amino acids 119, 120, 122, 200, 202-5, 207, 324-7, 369, 419, 421-3, 432-434, 436-8 of gp120. 48d was inhibited to 2% of control by 4 μM HNG-156 using ELISA.

The binding site for conformational antibody mAb 17b comprises amino acids 119, 120, 122, 200, 202-5, 419-23, 432-434, 437. 17b binding to gp120_{YU2} was inhibited to completion by 400 nM UM24 using ELISA.

Thus, HNG-156 and UM24 inhibit binding of antibodies that do not share a common epitope as a group, suggesting that inhibition is due to structuring a conformation of gp120 that precludes productive antibody binding.

### 2. Linear Antibodies

The binding site for linear antibody mAb ID6 linear Epitope comprises aa. 90-99 of gp120.

ID6 binding to gp120YU2 was inhibited to 96% of control by 10 uM UM24. Green: <10% change, blue: >10% enhancement, yellow: >10% inhibition.

The binding site for linear antibody mAb 11/65 comprises aa. 102-121 of gp120. This mAb bound at 102% of control with 10 uM UM24.

The binding site for linear antibody mAb 38.1 (Cordell et al., 1991, Virology 185:72-79; McKeating et al., 1992, Virology 190:134-142) comprises aa. 427, 430, 433, 435, 438, 482-484 of gp120. This antibody bound at 108% of control with 10 μM UM24.

The binding site for linear antibody mAb 2D7-V4 comprises aa. 394-404 of gp120. This antibody was inhibited to 85% by 100 μM UM24.

The binding site for linear antibody mAb F425-b4e8 linear comprises aa. 1309, P313, R315, F317 of gp120. This antibody bound at an average Of 110% of control with 100 μM UM24.

The binding site for linear antibody mAb 11/4C comprises aa. 161-171 on V1/V2 of gp120. This antibody bound at 116% of control with 1 mM UM24.

The binding site for linear antibody mAb 11/68b (McKeating et al., 1993, J. Virology 67:4932-4944) disulphide dependent Epitope comprises aa. 179-180, 183-1855, 191-193 of gp120. This mAb bound at 114% of control with 100 μM UM24.

The binding site for linear antibody mAb CRA3 (McKeating et al., 1993, J. Virology 67:4932-4944) disulphide dependent Epitope compmrises aa. 176-177, 179-180, 183-184, 192-194 of gp120. This antibody was inhibited to 84% by 100 μM UM24.

The binding site for linear antibody mAb CRA4 (McKeating et al., 1993, J. Virology Aug. 67:4932-4944) disulphide dependent Epitope comprises aa. 152-3, 176-7, 179-80, 183-4, 192-4 of gp120. Binding of this mAb was enhanced to 122% by 100 μM UM24.

The linear antibody mapping data help reveal sites of the inner domain that likely lie outside the peptide binding, due to non-competition. Epitopes 90-99 and 102-121 which contained amino acids previously thought to be in the HNG/UM footprint (K97 and E102) were not inhibited by LTM24. This localizes the UM24 epitope to regions of the inner domain near the CD4 binding site (D474 and R476) based on other mutagenesis data. Some disulphide-dependent epitopes on the V1/V2 loop and the b4e8 epitope on the V3 loop were made slightly more available by UM24 binding. This may indicate a conformational effect of peptide binding on these loops, as the V1V2 epitopes were previously seen to be affected by mutations in the CD4 binding site (Moore, 1993, J. Virol 67:6136-6151), and the b4e8 antibody was seen to structure the V3 loop in a unique way as to induce an a, rather than a 13-turn and shift the interactions at the tip of the loop based on crystal structure (Bell, 2008, J. Mol. Biol. 375:969-978).

### Experimental Example 2

### Antisera Against a Mixture of HIV-1 _{YU2} gp120 and the Dual Site Antagonist HNG-156 Show Neutralizing Activity for gp120 Binding to sCD4

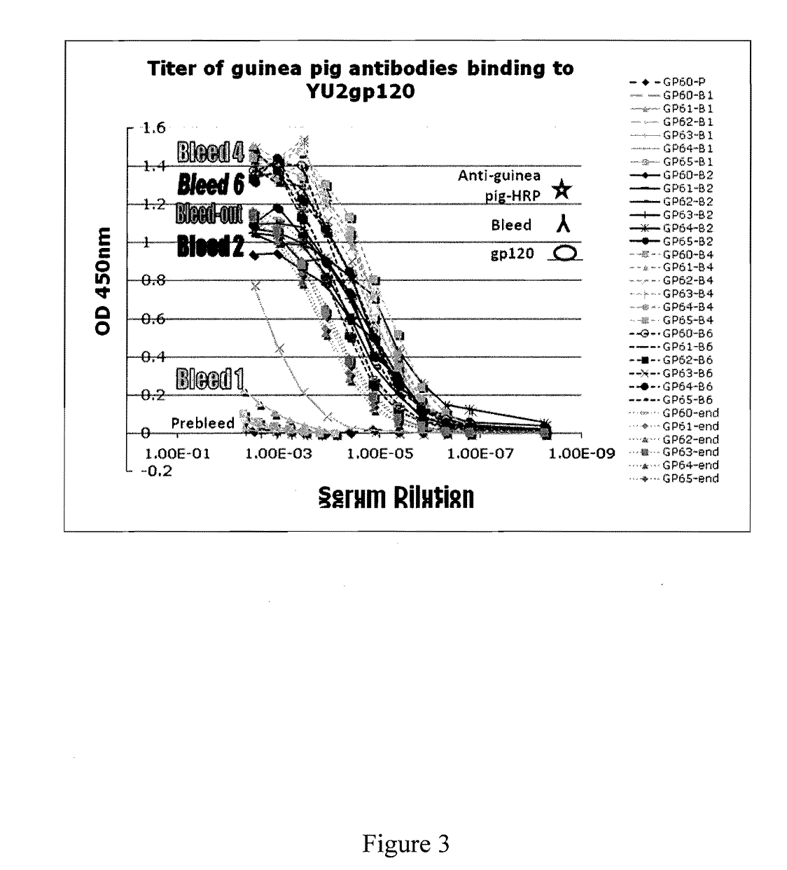

In an initial set of immunizations in guinea pigs (2 animals per immunogen), sera for immunizations with HIV-1_{YU2}gp120 alone versus HIV-1_{YU2}gp120 mixed with either the allosteric inhibitor HNG-156 or with a cyanovirin-12p1 allosteric inhibitor chimera denoted L5 were compared. Antisera from all guinea pigs contained antibodies to gp120, as shown by the representative data in FIG. 3.

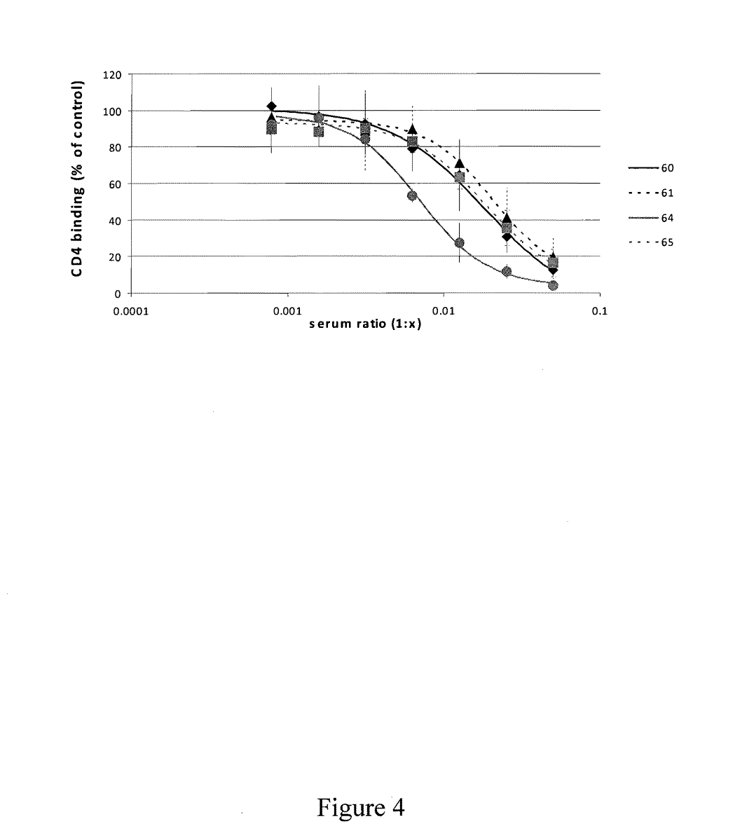

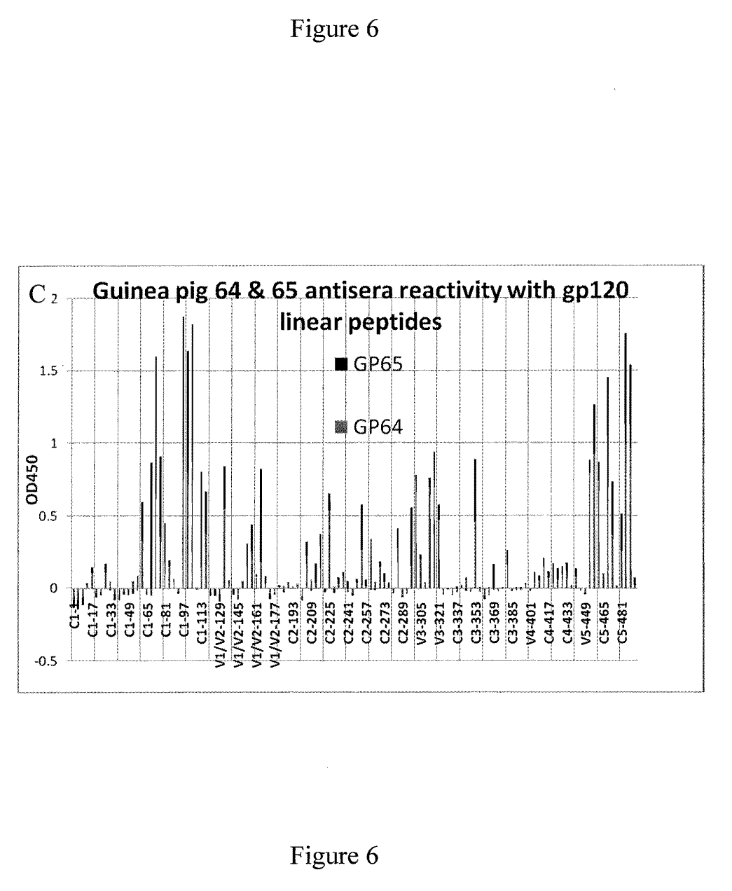

Different ELISA configurations were used to confirm neutralization activities. By ELISA analysis of CD4 binding to HIV-1_{YU2}gp120, all immunogens yielded sera with specific CD4-binding neutralization activity. Sera from one of the two [HIV-1_{YU2}gp120+HNG-156]-immunized guinea pigs were more CD4-neutralizing than the others when observed over several bleeds, and the enhanced CD4-neutralizing activity in this case increased with successive bleeds. Representative data for bleed 6 sera are shown in FIG. 4, along with the assay configuration used. Serum from GP 60 showed an almost equal competition activity to that for GP64 in the end-bleed though not so in earlier bleeds (FIG. 6A-C).

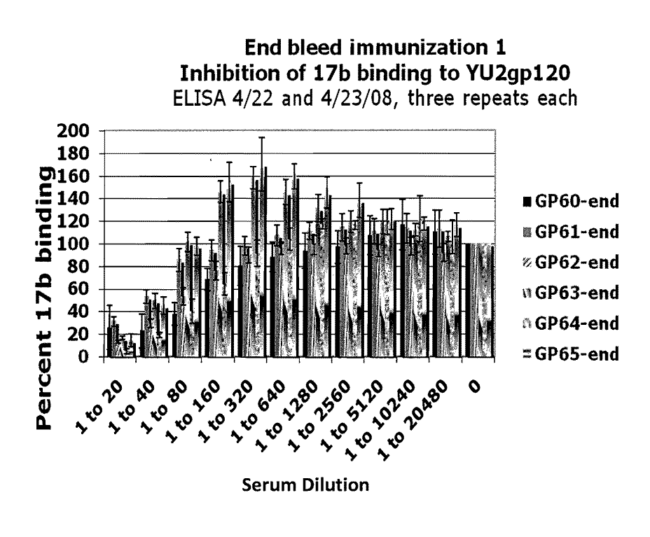

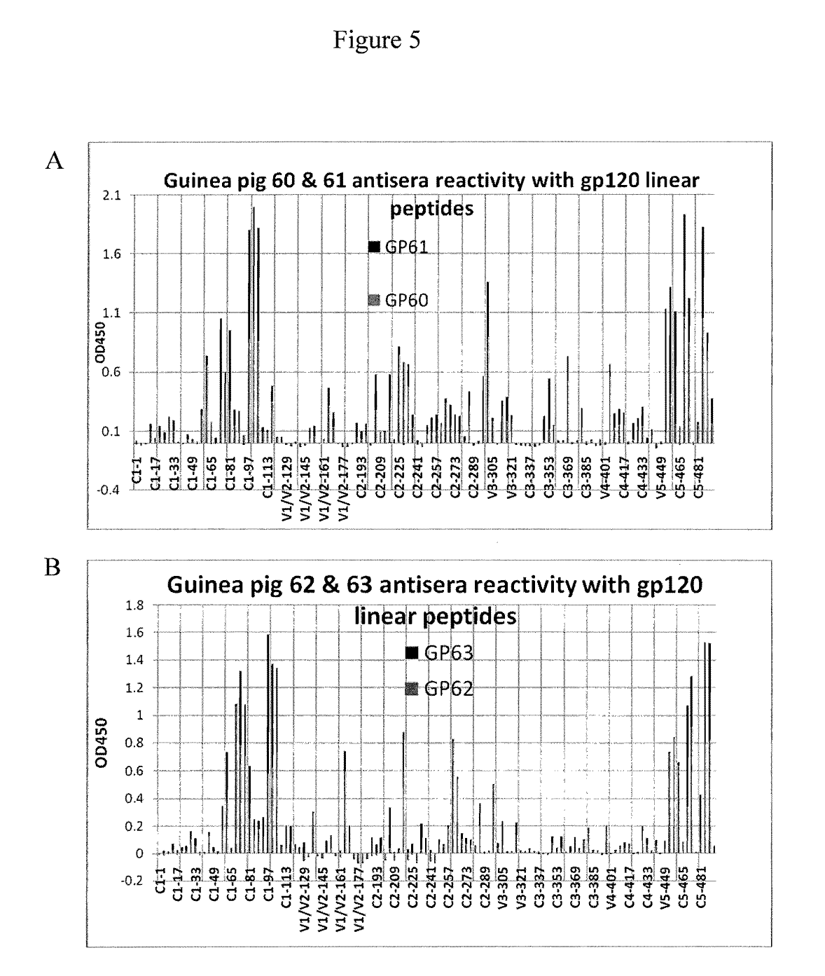

Neutralization of 17b binding, indicative of possible co-receptor site neutralization, also was observed in [HIV-1_{YU2}gp120+HNG-156] antisera as well as other sera (FIG. 5). Interestingly, bleeds from [HIV-1_{YU2}gp120+HNG-156] (both guinea pigs 64 and 65; FIG. 6C) and [HIV-1_{YU2}gp120+L5] (guinea pig 63) antisera showed an enhancement of 17b binding at intermediate serum dilutions. Without wishing to be bound by any particular theory, it is possible that some of the antibodies in these sera may mimic CD4, which is known to enhance 17b binding.

### Experimental Example 3

### Neutralization of Pseudoviral Cell Infection

Antisera from guinea pigs immunized with [HIV-1_{YU2}gp120+HNG-156] appear to show more consistent neutralizing activity of pseudoviral cell infection, including against “Tier 2” viruses (Table 1).

TABLE 1
Neutralization in TZM-bl cells with guinea pig antisera from immunization of HIV-lYU2gp120
alone (animals 60, 61) or in combination with L5 (animals 62, 63) or HNG-156 (animals 64, 65). Virus
stocks used were: MN (H9-grown, ID#792); SF162.LS (293T pseudovirus, ID#1718); Bal.26 (293T
pseudovirus, ID#680); 6535.3 (293T pseudovirus, ID #1778); QH0692.42 (293T pseudovirus, ID#1800);
PVO.4 (293T pseudovirus, ID#912); SVA-MLV (293T pseudovirus, ID#922).

| | | | ID50 in TZM-b1 cells1 | | | | | | |
|---|---|---|---|---|---|---|---|---|---|
| Animal | Bleed | Bleed | | | | | | | SVA- |
| ID | date | No. | MN | SF162.LS | Bal.26 | 6535.3 | QH0692.42 | PVO.4 | MLV |
| 60 | Aug. 23, 2007 | Prc | 22 | <20 | <20 | <20 | 28 | <20 | 25 |
| | Nov. 5, 2007 | 4 | 2,428 | 17,259 | 1,276 | 272 | 66 | <20 | <20 |
| | Feb. 1, 2008 | 6 | 3,401 | 23,436 | 1,503 | 239 | 59 | <20 | <20 |
| 61 | Aug. 23, 2007 | Prc | 33 | <20 | <20 | 22 | 34 | <20 | 27 |
| | Nov. 5, 2007 | 4 | 205 | 79 | <20 | 31 | 40 | <20 | 30 |
| | Feb. 1, 2008 | 6 | 380 | 132 | 39 | 90 | 57 | 25 | 44 |
| 62 | Aug. 23, 2007 | Prc | 26 | <20 | <20 | <20 | 26 | <20 | 30 |
| | Nov. 5, 2007 | 4 | 224 | 34 | 26 | 50 | 44 | <20 | 73 |
| | Feb. 1, 2008 | 6 | 105 | 35 | 31 | 81 | 66 | 22 | 47 |
| 63 | Aug. 23, 2007 | Prc | 23 | <20 | <20 | <20 | 29 | <20 | 29 |
| | Nov. 5, 2007 | 4 | 38 | 10,763 | <20 | 221 | 30 | <20 | 30 |
| | Feb. 1, 2008 | 6 | <20 | 12,524 | 34 | 612 | 36 | <20 | 34 |
| 64 | Aug. 23, 2007 | Prc | 34 | <20 | <20 | <20 | 28 | <20 | 39 |
| | Nov. 5, 2007 | 4 | 658 | 4,993 | 449 | 180 | 62 | <20 | 34 |
| | Feb. 1, 2008 | 6 | 2,035 | 4,894 | 370 | 293 | 134 | 56 | 47 |
| 65 | Aug. 23, 2007 | Prc | 31 | 22 | <20 | <20 | 31 | <20 | 26 |
| | Nov. 5, 2007 | 4 | 251 | 11,981 | 22 | 242 | 33 | <20 | 30 |
| | Feb. 1, 2008 | 6 | 284 | 20,360 | 28 | 170 | 32 | <20 | 21 |

¹Values are the sample dilution at which relative luminescence units (RLUs) were reduced 50% compared to virus control wells (no test sample).

These data show significant neutralization with both (2 of 2) [HIV-l_{YU2}gp120 +HNG-156] guinea pig sera, versus 1 of 2 guinea pigs given the other immunogens. Neutralization with [HIV-1_{YU2}gp120 +HNG-156] sera was observed with both Tier 1 (MN, SF162.LS and BaL26) and Tier 2 (,6535.3 and QH0692.42) viruses. Substantial neutralization activity was observed with guinea pig 60 (immunogen=HIV-1_{YU2}gp120 alone). Without wishing to be bound by any particular theory, this result may well be related to the finding that end-bleed sera from this guinea pig had increased CD4 neutralizing activity as determined by ELISA.

Table 2 depicts inhibition of viruses pseudotyped with HIV Env using a Tzm-bl luciferase assay using sera from rabbits immunized with HNG-156 and UM24. The sera was mixed with pseudovirus, which was later added to cells containing receptors for gp120 (CD4, CCR5 and CXCR4.) Viral entry was quantitated by light readout from luciferase expressed by infected cells. Rabbits were immunized with the following constructs: gp120 (Rabbits 85-88, gp120+HNG156 (non-covalent mixture) (rabbits 89-92), gp140-FOLDON trimeric protein (Rabbits 93-96), gp140-FOLDON+HNG-156 non covalent mixture (Rabbits 97-00). Pseudoviral neutralization by rabbit antisera showed slight advantages conferred by the non-covalent complexes versus protein alone in Tier 1 viruses (MN, SF162 and BaL26). However, neutralization capacity did not extend to the harder to neutralize Tier 2 strains (6535.3, QH0692.42, PVO.4 and YU2). SVA-MLV (Murine Leukemia Virus) was the specificity control.

TABLE 2

| | | | ID50 in TZM-bl cells¹ | | | | | | | |
|---|---|---|---|---|---|---|---|---|---|---|
| | Bleed | | | | | | | | | SVA- |
| Animal ID | date | Bleed | MN | SF162.LS | Bal.26 | 6535.3 | QH0692.42 | PVO.4 | YU2 | MLV |
| gp 120-R85 | Mar. 30, 2009 | Pre | <20 | <20 | <20 | <20 | <20 | <20 | <20 | <20 |
| | Jul. 1, 2009 | 6 | 35 | 1073 | 136 | <20 | <20 | <20 | <20 | <20 |
| gp120-R86 | Mar. 30, 2009 | Pre | 35 | <20 | <20 | <20 | <20 | <20 | <20 | <20 |
| | Jul. 1, 2009 | 6 | 42 | 1482 | 155 | <20 | <20 | <20 | <20 | <20 |
| gp120-R87 | Mar. 30, 2009 | Pre | 33 | <20 | <20 | <20 | <20 | <20 | <20 | 29 |
| | Jul. 1, 2009 | 6 | 22 | 1252 | 249 | <20 | <20 | <20 | <20 | <20 |
| gp120-R88 | Mar. 30, 2009 | Pre | 25 | <20 | <20 | <20 | <20 | <20 | <20 | <20 |
| | Jul. 1, 2009 | 6 | 23 | 139 | <20 | 37 | <20 | <20 | <20 | <20 |
| gp120+HNG-R89 | Mar. 30, 2009 | Pre | 32 | <20 | <20 | <20 | <20 | <20 | <20 | <20 |
| | Jul. 1, 2009 | 6 | 81 | 974 | 151 | <20 | <20 | <20 | <20 | <20 |
| gp120+HNG-R90 | Mar. 30, 2009 | Pre | 34 | <20 | <20 | <20 | <20 | <20 | <20 | <20 |
| | Jul. 1, 2009 | 6 | 1150 | 15898 | 340 | <20 | <20 | <20 | <20 | <20 |
| gp120+HNG-91 | Mar. 30, 2009 | Pre | 22 | <20 | <20 | <20 | <20 | <20 | <20 | <20 |
| | Jul. 1, 2009 | 6 | 26 | 1990 | 115 | <20 | <20 | <20 | <20 | <20 |
| gp120+HNG-R92 | Mar. 30, 2009 | Pre | 24 | <20 | <20 | <20 | <20 | <20 | <20 | <20 |
| | Jul. 1, 2009 | 6 | 40 | 8969 | 510 | <20 | <20 | <20 | <20 | <20 |
| gp140-R93 | Mar. 30, 2009 | Pre | 22 | <20 | <20 | <20 | <20 | <20 | <20 | <20 |
| | Jul. 1, 2009 | 6 | 346 | 826 | <20 | 63 | <20 | <20 | <20 | <20 |
| gp140-R94 | Mar. 30, 2009 | Pre | 37 | <20 | <20 | <20 | <20 | <20 | <20 | <20 |
| | Jul. 1, 2009 | 6 | 118 | 2269 | 53 | <20 | <20 | <20 | <20 | <20 |
| gp140-R95 | Mar. 30, 2009 | Pre | 25 | <20 | <20 | <20 | <20 | <20 | <20 | <20 |
| | Jul. 1, 2009 | 6 | 43740 | 7706 | 243 | 24 | <20 | <20 | <20 | <20 |
| gp140-R96 | Mar. 30, 2009 | Pre | 25 | <20 | <20 | <20 | <20 | <20 | <20 | 24 |
| | Jul. 1, 2009 | 6 | 3061 | 1111 | 108 | <20 | <20 | <20 | <20 | <20 |
| gp140+HNG-R97 | Mar. 30, 2009 | Pre | 26 | <20 | <20 | <20 | <20 | <20 | <20 | <20 |
| | Jul. 1, 2009 | 6 | 253 | 7987 | 180 | <20 | <20 | <20 | <20 | <20 |
| gp140+HNG-R98 | Mar. 30, 2009 | Pre | <20 | <20 | <20 | <20 | <20 | <20 | <20 | <20 |
| | Jul. 1, 2009 | 6 | 116 | 211 | <20 | <20 | <20 | <20 | <20 | <20 |
| gp140+HNG-R99 | Mar. 30, 2009 | Pre | 25 | <20 | <20 | <20 | <20 | <20 | <20 | <20 |
| | Jul. 1, 2009 | 6 | 197 | 3956 | 66 | <20 | <20 | <20 | <20 | <20 |
| gp140+HNG-R00 | Mar. 30, 2009 | Pre | <20 | <20 | <20 | <20 | <20 | <20 | <20 | <20 |
| | Jul. 1, 2009 | 6 | 253 | 11771 | 866 | 34 | <20 | <20 | <20 | <20 |

¹Values are the sample dilution at which relative luminescence units (RLUs) were reduced 50% compared to virus control wells (no test sample). Bolded values are >3 × background. Virus stocks: MN (H9-grown, ID#1124); SF162.LS (293T-PV, ID#2636); Bal.26 (293T-PV, ID#723); 6535.3 (293T-PV, ID#2841); QH0692.42 (293T-PV, ID#1997); PVO.4 (293T-PV, ID#1312); SVA-MLV (293T-PV, ID#923).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of obtaining a substantially pure population of neutralizing antibodies that prevent, inhibit, or block HIV-1 gp120 from binding to a CD4 T cell receptor, said method comprising the steps of:
a. administering to a subject an effective amount of a composition comprising an allosteric dual antagonist in combination with HIV-1 gp120, wherein when said composition is administered to said subject, said subject produces neutralizing antibodies that bind to said HIV-1 gp120;
b. isolating from the serum of said subject said substantially pure population of said neutralizing antibodies.

2. The method of claim 1, wherein said allosteric dual antagonist is selected from the group consisting of a peptide triazole conjugate, HNG-156, a fragment of HNG-156, UM24, a recombinant L5 cyanovirin-N-12p1 chimera, a small molecule, a peptide, or any combination thereof.

3. The method of claim 1, wherein said subject is a mammal.

4. The method of claim 3, wherein said mammal is a human.

5. A method of exposing a novel antigenic epitope present on the HIV-1 gp120 to immune surveillance in a subject, said method comprising administering to said subject a pharmaceutical composition comprising an immunogen, wherein said immunogen comprises an allosteric dual antagonist, wherein when said allosteric dual antagonist contacts said HIV-1 gp120, said allosteric dual antagonist induces a conformational change in said HIV-1 gp120, thereby exposing a novel antigenic epitope present on said HIV-1 gp120 to said immune surveillance.

6. The method of claim 5, wherein said allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof.

7. The method of claim 5, wherein said subject is a mammal.

8. The method of claim 7, wherein said mammal is a human.

9. A method of preventing HIV-1 infection in a subject, said method comprising administering to said subject a therapeutically effective amount of a pharmaceutical composition comprising an immunogen to a subject exposed to HIV-1 or at risk of being exposed to HIV-1, wherein said immunogen comprises an allosteric dual antagonist.

10. The method of claim 9, wherein said allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof.

11. The method of claim 9, wherein said subject is a mammal.

12. The method of claim 11, wherein said mammal is a human.

13. A method of treating HIV-1 infection in a subject, said method comprising administering to said subject a therapeutically effective amount of a pharmaceutical composition to a subject infected with HIV-1, wherein said composition comprises an allosteric dual antagonist and HIV-1 gp120.

14. The method of claim 13, wherein said allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof.

15. The method of claim 14, wherein said subject is a mammal.

16. The method of claim 15, wherein said mammal is a human.

17. A method of treating HIV-1 infection of a subject, said method comprising administering to said subject a therapeutically effective amount of a neutralizing antibody to said subject, wherein said neutralizing antibody neutralizes the interaction of HIV-1 gp120 and CD4 receptors.

18. The method of claim 17, wherein said subject is a mammal.

19. The method of claim 18, wherein said mammal is a human.

20. A method of inhibiting the interaction of HIV-1 gp120 and a CD4 T cell receptor in a subject infected with HIV-1, said method comprising administering to said subject a therapeutically effective amount of a pharmaceutical composition comprising an immunogen to said subject infected with HIV-1, wherein said composition comprises an allosteric dual antagonist and HIV-1 gp120.

21. The method of claim 20, wherein said allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof.

22. The method of claim 21, wherein said subject is a mammal.

23. The method of claim 22, wherein said mammal is a human.

24. A method of inhibiting the interaction of HIV-1 gp120 and a CD4 T cell receptor in a subject infected with HIV-1, said method comprising administering to said subject a therapeutically effective amount of a neutralizing antibody to said subject, wherein said neutralizing antibody neutralizes the interaction of HIV-1 gp120 and said CD4 T-cell receptors.

25. The method of claim 24, wherein said subject is a mammal.

26. The method of claim 25, wherein said mammal is a human.

27. A pharmaceutical composition comprising an immunogen, wherein said immunogen comprises an allosteric dual antagonist and an HIV-1 gp120.

28. The immunogen of claim 27, wherein said allosteric dual antagonist selected from the group consisting of a peptide triazole conjugate, HNG-156, a fragment of HNG-156, UM24, a recombinant L5 cyanovirin-N-1p12 chimera, a small molecule, a peptide, or any combination thereof.

29. A pharmaceutical composition comprising a neutralizing antibody, wherein said neutralizing antibody is made in response to an immunogen, wherein said immunogen comprises an allosteric dual antagonist, wherein said neutralizing antibody inhibits the interaction of HIV-1 gp120 and a CD4 T cell receptor.

30. A method of exposing a novel antigenic epitope present on the HIV-1 gp120 to immune surveillance in a subject, said method comprising administering to said subject a pharmaceutical composition comprising an allosteric dual antagonist, wherein when said allosteric dual antagonist contacts said HIV-1 gp120, said allosteric dual antagonist induces a conformational change in said HIV-1 gp120, thereby exposing a novel antigenic epitope present on said HIV-1 gp120 to said immune surveillance.

31. The method of claim 30, wherein said allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof.

32. The method of claim 31, wherein said allosteric dual antagonist is administered in combination with exogenous HIV-1 gp120.

33. The method of claim 31, where said allosteric dual antagonist is administered without exogenous HIV-1 gp120.

34. The method of claim 31, wherein said subject is a mammal.

35. The method of claim 31, wherein said mammal is a human.

36. A method of preventing HIV-1 infection in a subject, said method comprising administering to said subject a therapeutically effective amount of a pharmaceutical composition comprising an immunogen to a subject exposed to HIV-1 or at risk of being exposed to HIV-1, wherein said immunogen comprises an allosteric dual antagonist.

37. The method of claim 36, wherein said allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof.

38. The method of claim 37, wherein said allosteric dual antagonist is administered in combination with exogenous HIV-1 gp120.

39. The method of claim 37, where said allosteric dual antagonist is administered without exogenous HIV-1 gp120.

40. The method of claim 37, wherein said subject is a mammal.

41. The method of claim 37, wherein said mammal is a human.

42. A method of treating HIV-1 infection in a subject, said method comprising administering to said subject a therapeutically effective amount of a pharmaceutical composition to a subject infected with HIV-1, wherein said composition comprises an allosteric dual antagonist and HIV-1 gp120.

43. The method of claim 42, wherein said allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof.

44. The method of claim 43, wherein said allosteric dual antagonist is administered in combination with exogenous HIV-1 gp120.

45. The method of claim 43, where said allosteric dual antagonist is administered without exogenous HIV-1 gp120.

46. The method of claim 43, wherein said subject is a mammal.

47. The method of claim 43, wherein said mammal is a human.

48. A composition for inhibiting binding of HIV-1 gp120 to a host CD4 receptor, said composition comprising an allosteric dual antagonist covalently linked to gp120.

49. The composition of claim 48, wherein said allosteric dual antagonist is selected from the group consisting of a peptide triazole conjugate, HNG-156, a fragment of HNG-156, UM24, a recombinant L5 cyanovirin-N-1p12 chimera, a small molecule, a peptide, or any combination thereof.

50. The composition of claim 48, wherein said host is a mammal.

51. The composition of claim 50, wherein said mammal is a human.


SEQUENCE LISTING (8 sequences)

SEQ ID NO: 1 - protein, 12 aa, Artificial Sequence
  Chemically synthesized
RINNIPWSEAMM

SEQ ID NO: 2 - protein, 7 aa, Artificial Sequence
  Chemically synthesized
INNIPWS

SEQ ID NO: 3 - protein, 12 aa, Artificial Sequence
  Chemically synthesized
RINNIPWSEAMM

SEQ ID NO: 4 - protein, 101 aa, Artificial Sequence
  Chemically synthesized
LGKFSQTCYNSAIQGSVLTSTCERTNGGYNTSSIDLNSVIENVDGSLKWQPSNFIETCRNTQLAGSSELAAECKTRAQQFVSTKINLDDHIANIDGTLKYE

SEQ ID NO: 5 - DNA, 302 bp, Artificial Sequence
  Chemically synthesized
cttgtaaattctcccagacctgctacaactccgctatccagggttccgttctgacctccacctgcgaacgtaccaacggtggttacaacacctcctccatcgacctgaactccgttatcgaaaacgttgacggttccctgaaatggcagccgtccaacttcatcgaaacctgccgtaacacccagctggctggttcctccgaactggctgctgaatgcaaaacccgtgctcagcagttcgtttccaccaaaatcaacctggacgaccacatcgctaacatcgacggtaccctgaaatacgaa

SEQ ID NO: 6 - protein, 12 aa, Artificial Sequence
  Chemically synthesized
RINNIXWSEAMM

SEQ ID NO: 7 - protein, 12 aa, Artificial Sequence
  Chemically synthesized
RINNIXWESAMM

SEQ ID NO: 8 - protein, 7 aa, Artificial Sequence
  Chemically synthesized
XNNIXWS